(19) United States
(12) Patent Application Publication
(10) Pub. No.: US 2015/0246061 A1
(43) Pub. Date: Sep. 3, 2015

(54) VITAMIN D ANALOGUES FOR THE TREATMENT OF A NEUROLOGICAL DISORDER

(71) Applicant: CELUS PHARMACEUTICALS, INC., San Jose, CA (US)

(72) Inventor: Kjell Stenberg, Akers Styckebruk (SE)

(21) Appl. No.: 14/423,960

(22) PCT Filed: Oct. 18, 2013
(86) PCT No.: PCT/US13/65766

§ 371 Date: Feb. 25, 2015

Related U.S. Application Data
(60) Provisional application No. 61/716,377, filed on Oct. 19, 2012.

(51) Int. Cl. A61K 31/59; A61K 31/192; A61K 31/198; A61K 31/195; A61K 31/13; A61K 31/445

(52) U.S. Cl. CPC A61K 31/59; A61K 31/13; A61K 31/445; A61K 31/198; A61K 31/195; A61K 31/192

(57) ABSTRACT

Disclosed herein are methods that use a vitamin D analogue to treat a neurological disorder. Additionally, pharmaceutical compositions comprising a vitamin D analogue and a neurotherapeutic agent are disclosed, and methods of using the same. The pharmaceutical compositions and methods of the invention are useful for the treatment of a neurological disorder, for example, Alzheimer's disease, multiple sclerosis, Parkinson's disease, epilepsy, neuropathic pain, or other conditions that affect the central or peripheral nervous system of a subject.

### CROSS REFERENCE

This application claims priority to U.S. Provisional Patent Application No. 61/716,377, filed Oct. 19, 2012, which is herein incorporated by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND OF THE INVENTION

A neurological disorder is an abnormal condition, disorder, or disease of the central and/or peripheral nervous system of a living organism. A neurological disorder may affect almost any organism that possesses a nervous system and there are many types of neurological disorders that are presented in subjects. Indeed, the World Health Organization (WHO) reported in 2007 that up to one billion people suffer from a neurological disorder at any given time. The WHO also reported that this statistic is expected to increase further as the population continues to age—it is generally accepted that neurological disorders are more common in older subjects. Neurological disorders can range in severity from mild to extremely severe. For example, a generally non life-threatening, but common neurological disorder is recurrent migraines, manifested by headaches that occur repetitively in a certain region of a subject's head. In contrast, the prognosis of patients diagnosed with Amytrophic lateral sclerosis (ALS) is generally terminal. In ALS, nerve cells waste away and can no longer communicate with muscles, to the point where an afflicted subject can no longer breathe on his or her own. Neurological disorders may be complex and may often develop gradually. Moreover, the mechanisms predominately driving the course of a disease may change over time with disease progression, resulting in a set of moving, varied targets possibly useful for therapeutic intervention.

Elocalcitol is a vitamin D analogue that may produce anti-proliferative and anti-inflammatory effects. As a result, elocalcitol has been studied as an experimental drug for a number of indications including overactive bladder, male infertility, chronic non-bacterial prostatitis, benign prostate hyperplasia, and osteoporosis. It is generally accepted that elocalcitol has been shown to be safe for adult humans for longer treatment periods up to a daily dose of 150 μg (Montorsi F. et al. “J Urol 2008; 179(suppl): 700; Abstract 2035).

### SUMMARY OF THE INVENTION

In some embodiments, the invention provides a method of treating a neurological disorder in a subject, the method comprising administering a therapeutically effective amount of a vitamin D analogue, or a pharmaceutically-acceptable salt thereof; to a subject in need or want thereof, wherein the dose of said vitamin D analogue is limited to a threshold under which hypercalcemia is not induced.

In some embodiments, the invention provides a pharmaceutical composition comprising: a) a vitamin D analogue, or a pharmaceutically-acceptable salt thereof; and b) a neurotherapeutic, or a pharmaceutically-acceptable salt thereof.

In some embodiments, the invention provides a method of treating a neurological disorder in a subject, the method comprising administering a therapeutically effective amount of a vitamin D analogue, or a pharmaceutically-acceptable salt thereof; and a therapeutically effective amount of a neurotherapeutic, or a pharmaceutically-acceptable salt thereof, to the subject, wherein the dose of said vitamin D analogue is limited to a threshold under which hypercalcemia is not induced.

### DETAILED DESCRIPTION OF THE INVENTION

The present invention relates to methods and pharmaceutical formulations for the treatment of a neurological disorder. Non-limiting examples of neurological disorders treatable by the methods of the present invention include Alzheimer's disease, multiple sclerosis, Parkinson's disease, epilepsy, and neuropathic pain. Such conditions may be ameliorated by the administration of a vitamin D analogue or a pharmaceutical composition of the invention comprising a vitamin D analogue and at least one neurotherapeutic compound.

One example of a vitamin D analogue suitable for use in the compositions or methods of the invention is elocalcitol, which is a synthetic, biologically active vitamin D analogue with modifications to the side chain and A ring. The chemical structure of elocalcitol is:

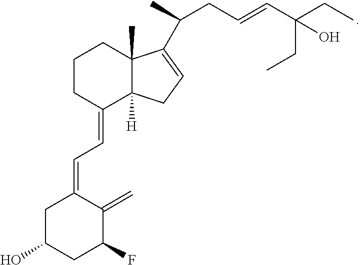 [SMILES: C=C1/C(=C\C=C2/CCC[C@]3(C)C([C@@H](C)C/C=C/C(O)(CC)CC)=CC[C@@H]23)C[C@@H](O)C[C@@H]1F]

Elocalcitol is a vitamin D analogue that may function as an inhibitor of Rho-associated protein kinase (ROCK) that may regulate the shape and movement of cells by acting on the cell cytoskeleton. Antiproliferative and anti-inflammatory effects may be observed with elocalcitol. Moreover, elocalcitol may be administered at therapeutic doses over prolonged periods without affecting calcium levels or bone structure. Due to its attractive attributes, elocalcitol has been studied as an experimental drug for a number of indications including overactive bladder, male infertility, chronic non-bacterial prostatitis, benign prostate hyperplasia, and osteoporosis. Elocalcitol has been shown to be safe for adult humans for longer treatment periods up to a daily dose of 150 μg (Montorsi F. et al. J Urol 2008; 179(suppl): 700; Abstract 2035). At doses higher than 150 μg per day, hypercalcemia, a common side-effect of prolonged use of a vitamin D analogue, may ensue.

A neurological disorder may be chronic once established and may, in whole or part, be due to inflammatory or, more generally, immunological pathways. In one example, the role of inflammatory pathways has been investigated for a number of neurological conditions, including Alzheimer's disease and Parkinson's disease. Long-term studies of patients with Alzheimer's disease and Parkinson's disease treated with non-steroidal anti-inflammatory drugs (NSAIDs) appeared to show reduced disease symptoms after sustained treatment (McGeer et al, Lancet, 1990: 1037). As a result of patient response to anti-inflammatory agents, one underlying cause of these diseases may be related to one or more inflammatory processes or pathways (Zhou et al, Science, 14 Nov. 2003: 1215-1217). In another example, one underlying cause of seizures in epileptic patients may be due to structural failures of a subject's blood-brain-barrier (BBB), wherein unwanted species are permitted entry into the brain. These structural failures may be linked to inflammation that results in disruption of the BBB. Moreover, recent research indicates that inflammatory events induced by nerve injury may play an important role in the pathogenesis of neuropathic pain (Tal, Curr Rev. Pain 1999;3 (6):440-446). In other disorders, an underlying cause of a neurological disorder is known to be autoimmune in nature, such as in the case of multiple sclerosis. In these diseases, a subject's own immune system attacks and damages structures critical to normal neurological function.

Elocalcitol may be beneficial in treating disorders that are associated with inflammatory events or activation of a subject's immune system (e.g. by genetic predispositions, as a response linked to an endogenous condition, or as a response to an external event such as injury). Therefore, it is possible that a neurological disorder that may be, in part or whole, linked to an inflammatory or an immunological response, may be treatable with elocalcitol. Combination therapy of elocalcitol and a disease-specific established therapy (or therapies) may also be beneficial.

Immunological and inflammatory processes may be inter-dependent and may be related through intermediate mediators (e.g. changes to one function or immune cell may affect another function or immune cell that, in turn, may affect another function or immune cell, and so on). Non-limiting examples of possible modes of action for elocalcitol that correspond to inflammatory pathways include inhibition of NFκB/AP-1, stimulation of PPARγ, inhibition of COX, Aβ phagocytosis, reduction of reactive oxygen species, inhibition or reduction of cytokine production, or inhibition or reduction of metalloproteases.

Additional non-limiting examples of possible modes of action possibly therapeutic with respect to a neurological disorder include neuroprotection (e.g., induction of neurotrophic factors such as nerve growth factor (NGF), brain-derived neurotrophic factor (BDNF), glial cell line-derived neurotrophic factor (GDNF); inhibition of Tau phosphorylation; increased production of neutrophines; inhibition of choline esterases), aspects of Aβ metabolism (e.g. stimulate Aβ degradation, inhibition of β and γ secretases, stimulation of a secretases, inhibition of Aβ oligomer formation, chelation of Aβ monomers), aspects of cholesterol homeostasis (e.g. reduction of cholesterol levels), or anti-oxidation (e.g. stimulate radical scavengers, increased production of anti-oxidants).

Compounds of the Invention

In some embodiments, a vitamin D analogue for use in a pharmaceutical composition or method of the invention is a vitamin D analogue of the formula:

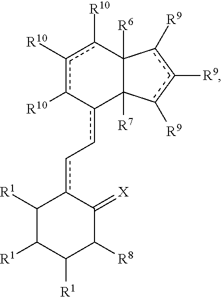

wherein:
each R¹, R⁶, R⁷, R⁸, R⁹ and R¹⁰ is independently H, halo, alkyl, alkenyl, alkynyl, aryl, aralkyl, hydroxyl, sulfhydryl, amino, nitro, cyano, alkoxy, an ester group, an amide group, a carbonate group, a carbamate group, or is a group of the formula:

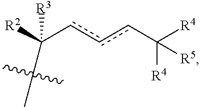

wherein:
each R², R³, R⁴, and R⁵ is independently H, halo, alkyl, aryl, or hydroxyl;
X is C(R¹)₂, O, or NR¹; and
each alkyl, alkenyl, alkynyl, aryl, aralkyl, amino, and alkoxy group is optionally substituted, or a pharmaceutically-acceptable salt thereof.

In some embodiments, one or more of R¹, R⁶, R⁷, R⁸, R⁹ and R¹⁰ is a group of the formula:

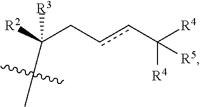

wherein each R², R³, R⁴, and R⁵ is independently H, halo, alkyl, aryl, or hydroxyl.

In some embodiments, a vitamin D analogue for use in a pharmaceutical composition or method of the invention is a vitamin D analogue of the formula:

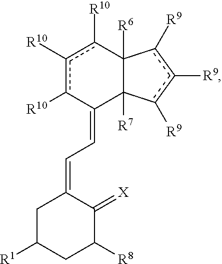

wherein:
each variable is as described previously, or a pharmaceutically-acceptable salt thereof.

In some embodiments, a vitamin D analogue for use in a pharmaceutical composition or method of the invention is a vitamin D analogue of the formula:

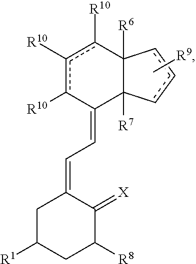

wherein:
each variable is as described previously, or a pharmaceutically-acceptable salt thereof.

In some embodiments, a vitamin D analogue for use in a pharmaceutical composition or method of the invention is a vitamin D analogue of the formula:

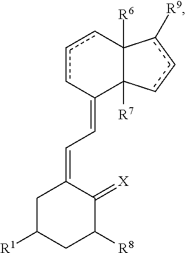

wherein:
each variable is as described previously, or a pharmaceutically-acceptable salt thereof.

In some embodiments, a vitamin D analogue for use in a pharmaceutical composition or method of the invention is a vitamin D analogue of the formula:

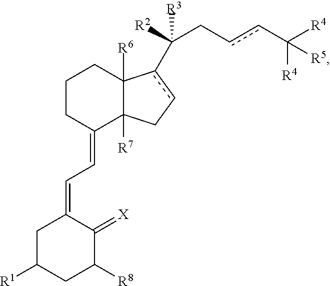

wherein:
each R¹, R⁶, R⁷, and R⁸ is independently H, halo, alkyl, alkenyl, alkynyl, aryl, aralkyl, hydroxyl, sulfhydryl, amino, nitro, cyano, alkoxy, an ester group, an amide group, a carbonate group, or a carbamate group;
each R², R³, R⁴, and R⁵ is independently H, halo, alkyl, aryl, or hydroxyl;
X is C(R¹)₂, O, or NR¹; and
each alkyl, alkenyl, alkynyl, aryl, aralkyl, amino, and alkoxy group is optionally substituted, or a pharmaceutically-acceptable salt thereof. In some embodiments, R¹ is H, halo, alkyl, or hydroxyl; R² is H or alkyl; R³ is H or alkyl; each R⁴ is independently H or alkyl; R⁵ is H, alkyl, or hydroxyl; R⁶ is H or alkyl; R⁷ is H or alkyl; R⁸ is H, halo, alkyl, or hydroxyl; and X is CH₂ or O.

In some embodiments, a vitamin D analogue for use in a pharmaceutical composition or method of the invention is a vitamin D analogue of the formula:

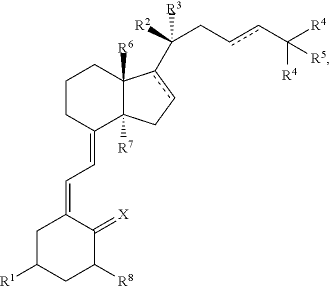

wherein:
each R¹, R⁶, R⁷, and R⁸ is independently H, halo, alkyl, alkenyl, alkynyl, aryl, aralkyl, hydroxyl, sulfhydryl, amino, nitro, cyano, alkoxy, an ester group, an amide group, a carbonate group, or a carbamate group;
each R², R³, R⁴, and R⁵ is independently H, halo, alkyl, aryl, or hydroxyl;
X is C(R¹)₂, O, or NR¹; and
each alkyl, alkenyl, alkynyl, aryl, aralkyl, amino, and alkoxy group is optionally substituted, or a pharmaceutically-acceptable salt thereof. In some embodiments, R¹ is H, halo, alkyl, or hydroxyl; R² is H or alkyl; R³ is H or alkyl; each R⁴ is independently H or alkyl; R⁵ is H, alkyl, or hydroxyl; R⁶ is H or alkyl; R⁷ is H or alkyl; R⁸ is H, halo, alkyl, or hydroxyl; and X is CH₂ or O.

In some embodiments, a vitamin D analogue for use in a pharmaceutical composition or method of the invention is a vitamin D analogue of the formula:

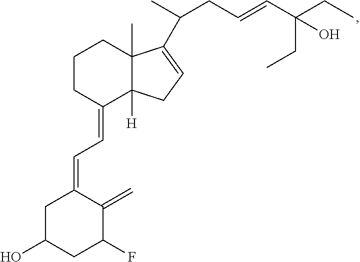 [SMILES: C=C1/C(=C\C=C2/CCCC3(C)C(C(C)C/C=C/C(O)(CC)CC)=CCC23)CC(O)CC1F]

or a pharmaceutically-acceptable salt thereof.

In some embodiments, a vitamin D analogue for use in a pharmaceutical composition or method of the invention is a vitamin D analogue of the formula:

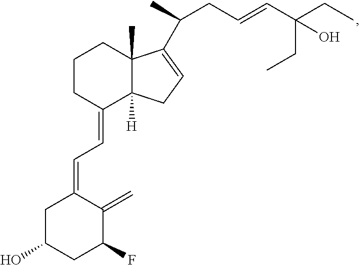 [SMILES: C=C1/C(=C\C=C2/CCC[C@]3(C)C([C@@H](C)C/C=C/C(O)(CC)CC)=CC[C@@H]23)C[C@@H](O)C[C@@H]1F]

or a pharmaceutically-acceptable salt thereof.

In some embodiments, a vitamin D analogue for use in a pharmaceutical composition or method of the invention is elocalcitol, or a pharmaceutically-acceptable salt thereof.

Non-limiting examples of vitamin D analogues suitable for use in the pharmaceutical compositions and methods of the invention include:

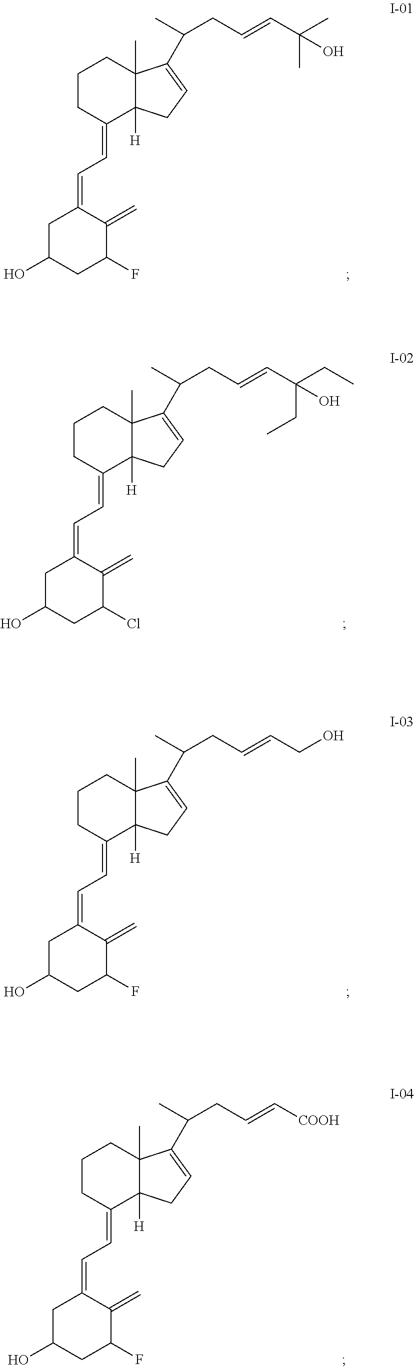 [SMILES: C=C1/C(=C\C=C2/CCCC3(C)C(C(C)C/C=C/C(C)(C)O)=CCC23)CC(O)CC1F ; C=C1/C(=C\C=C2/CCCC3(C)C(C(C)C/C=C/C(O)(CC)CC)=CCC23)CC(O)CC1Cl ; C=C1/C(=C\C=C2/CCCC3(C)C(C(C)C/C=C/CO)=CCC23)CC(O)CC1F ; C=C1/C(=C\C=C2/CCCC3(C)C(C(C)C/C=C/C(=O)O)=CCC23)CC(O)CC1F]

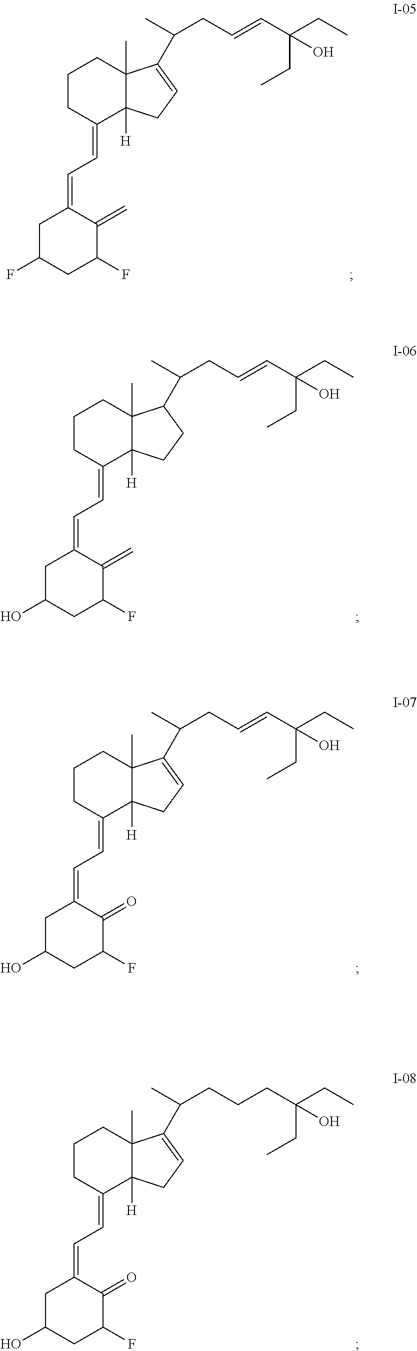 [SMILES: C=C1/C(=C\C=C2/CCCC3(C)C(C(C)C/C=C/C(O)(CC)CC)=CCC23)CC(F)CC1F ; C=C1/C(=C\C=C2/CCCC3(C)C2CCC3C(C)C/C=C/C(O)(CC)CC)CC(O)CC1F ; CCC(O)(/C=C/CC(C)C1=CCC2/C(=C/C=C3/CC(O)CC(F)C3=O)CCCC12C)CC ; CCC(O)(CC)CCCC(C)C1=CCC2/C(=C/C=C3/CC(O)CC(F)C3=O)CCCC12C]

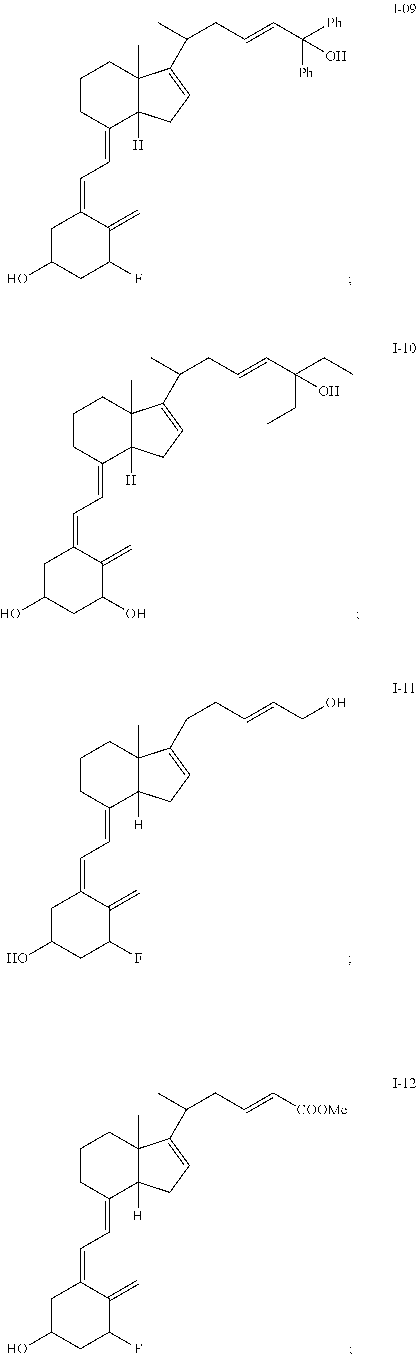 [SMILES: C=C1/C(=C\C=C2/CCCC3(C)C(C(C)C/C=C/C(O)(c4ccccc4)c4ccccc4)=CCC23)CC(O)CC1F ; C=C1/C(=C\C=C2/CCCC3(C)C(C(C)C/C=C/C(O)(CC)CC)=CCC23)CC(O)CC1O ; C=C1/C(=C\C=C2/CCCC3(C)C(CC/C=C/CO)=CCC23)CC(O)CC1F ; C=C1/C(=C\C=C2/CCCC3(C)C(C(C)C/C=C/C(=O)OC)=CCC23)CC(O)CC1F]

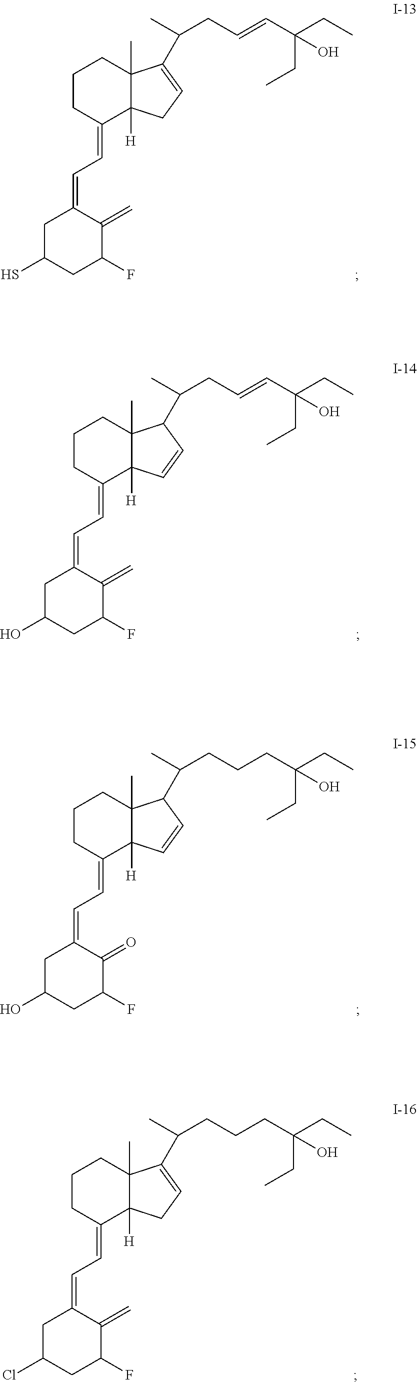 [SMILES: C=C1/C(=C\C=C2/CCCC3(C)C(C(C)C/C=C/C(O)(CC)CC)=CCC23)CC(S)CC1F ; C=C1/C(=C\C=C2/CCCC3(C)C2C=CC3C(C)C/C=C/C(O)(CC)CC)CC(O)CC1F ; CCC(O)(CC)CCCC(C)C1C=CC2/C(=C/C=C3/CC(O)CC(F)C3=O)CCCC21C ; C=C1/C(=C\C=C2/CCCC3(C)C(C(C)CCCC(O)(CC)CC)=CCC23)CC(Cl)CC1F]

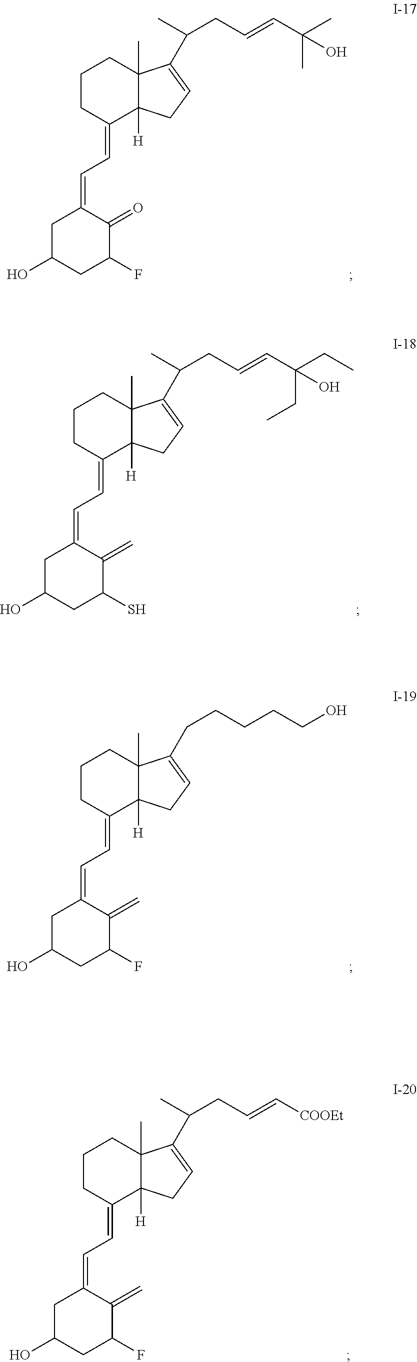 [SMILES: CC(C/C=C/C(C)(C)O)C1=CCC2/C(=C/C=C3/CC(O)CC(F)C3=O)CCCC12C ; C=C1/C(=C\C=C2/CCCC3(C)C(C(C)C/C=C/C(O)(CC)CC)=CCC23)CC(O)CC1S ; C=C1/C(=C\C=C2/CCCC3(C)C(CCCCCO)=CCC23)CC(O)CC1F ; C=C1/C(=C\C=C2/CCCC3(C)C(C(C)C/C=C/C(=O)OCC)=CCC23)CC(O)CC1F]

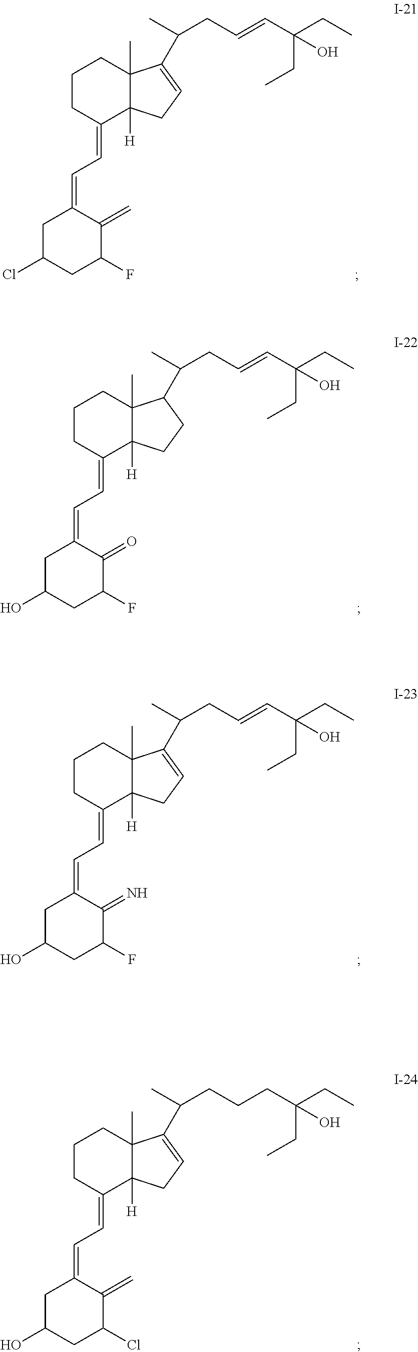 [SMILES: C=C1/C(=C\C=C2/CCCC3(C)C(C(C)C/C=C/C(O)(CC)CC)=CCC23)CC(Cl)CC1F ; CCC(O)(/C=C/CC(C)C1CCC2/C(=C/C=C3/CC(O)CC(F)C3=O)CCCC21C)CC ; CCC(O)(/C=C/CC(C)C1=CCC2/C(=C/C=C3/CC(O)CC(F)C3=N)CCCC12C)CC ; C=C1/C(=C\C=C2/CCCC3(C)C(C(C)CCCC(O)(CC)CC)=CCC23)CC(O)CC1Cl]

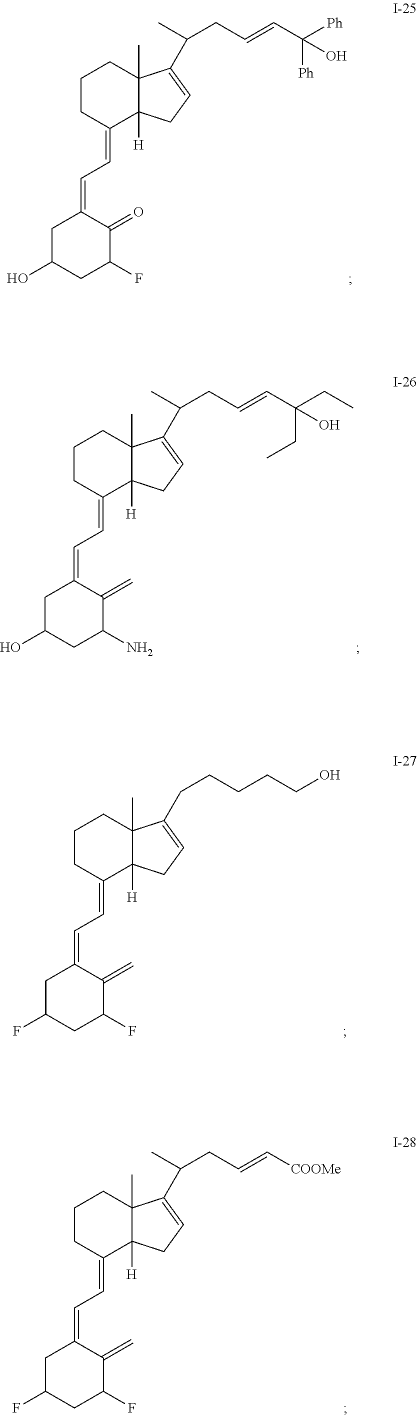 [SMILES: CC(C/C=C/C(O)(c1ccccc1)c1ccccc1)C1=CCC2/C(=C/C=C3/CC(O)CC(F)C3=O)CCCC12C ; C=C1/C(=C\C=C2/CCCC3(C)C(C(C)C/C=C/C(O)(CC)CC)=CCC23)CC(O)CC1N ; C=C1/C(=C\C=C2/CCCC3(C)C(CCCCCO)=CCC23)CC(F)CC1F ; C=C1/C(=C\C=C2/CCCC3(C)C(C(C)C/C=C/C(=O)OC)=CCC23)CC(F)CC1F]

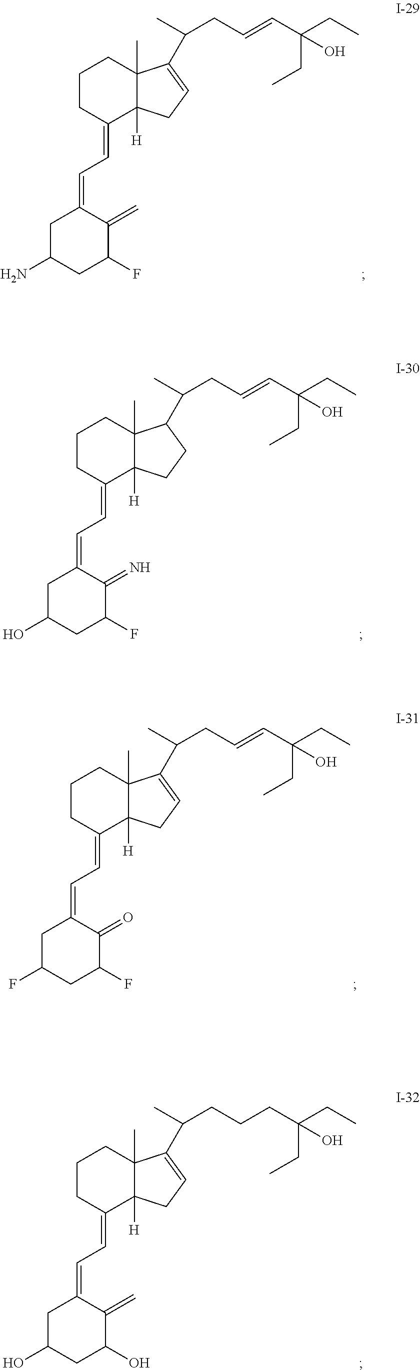 [SMILES: C=C1/C(=C\C=C2/CCCC3(C)C(C(C)C/C=C/C(O)(CC)CC)=CCC23)CC(N)CC1F ; CCC(O)(/C=C/CC(C)C1CCC2/C(=C/C=C3/CC(O)CC(F)C3=N)CCCC21C)CC ; CCC(O)(/C=C/CC(C)C1=CCC2/C(=C/C=C3/CC(F)CC(F)C3=O)CCCC12C)CC ; C=C1/C(=C\C=C2/CCCC3(C)C(C(C)CCCC(O)(CC)CC)=CCC23)CC(O)CC1O]

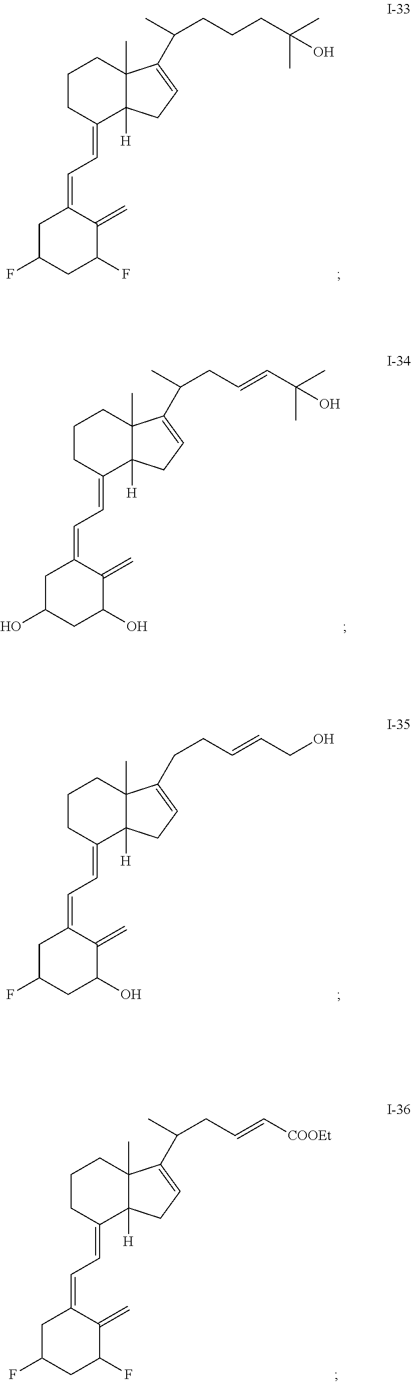 [SMILES: C=C1/C(=C\C=C2/CCCC3(C)C(C(C)CCCC(C)(C)O)=CCC23)CC(F)CC1F ; C=C1/C(=C\C=C2/CCCC3(C)C(C(C)C/C=C/C(C)(C)O)=CCC23)CC(O)CC1O ; C=C1/C(=C\C=C2/CCCC3(C)C(CC/C=C/CO)=CCC23)CC(F)CC1O ; C=C1/C(=C\C=C2/CCCC3(C)C(C(C)C/C=C/C(=O)OCC)=CCC23)CC(F)CC1F]

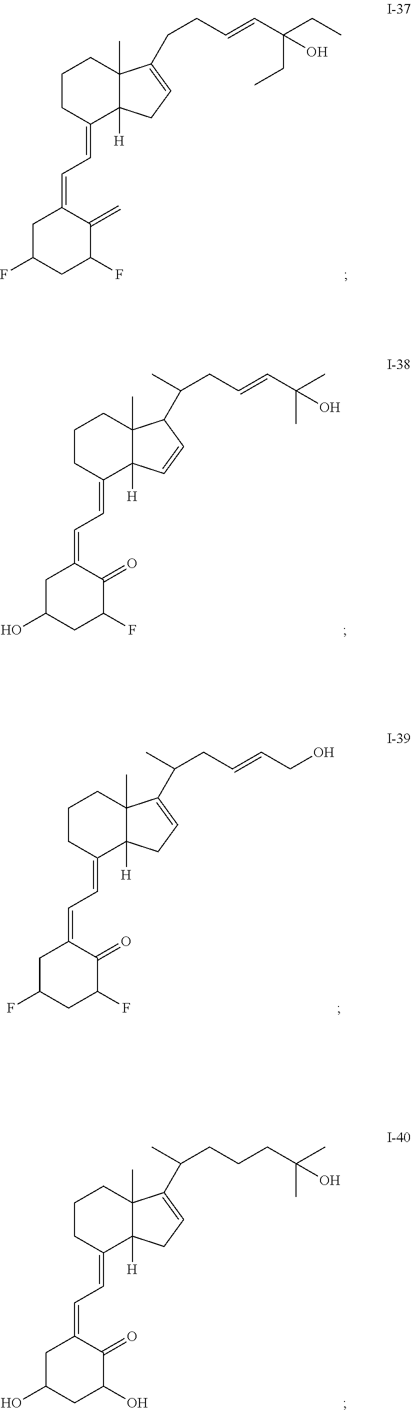 [SMILES: C=C1/C(=C\C=C2/CCCC3(C)C(CC/C=C/C(O)(CC)CC)=CCC23)CC(F)CC1F ; CC(C/C=C/C(C)(C)O)C1C=CC2/C(=C/C=C3/CC(O)CC(F)C3=O)CCCC21C ; CC(C/C=C/CO)C1=CCC2/C(=C/C=C3/CC(F)CC(F)C3=O)CCCC12C ; CC(CCCC(C)(C)O)C1=CCC2/C(=C/C=C3/CC(O)CC(O)C3=O)CCCC12C]

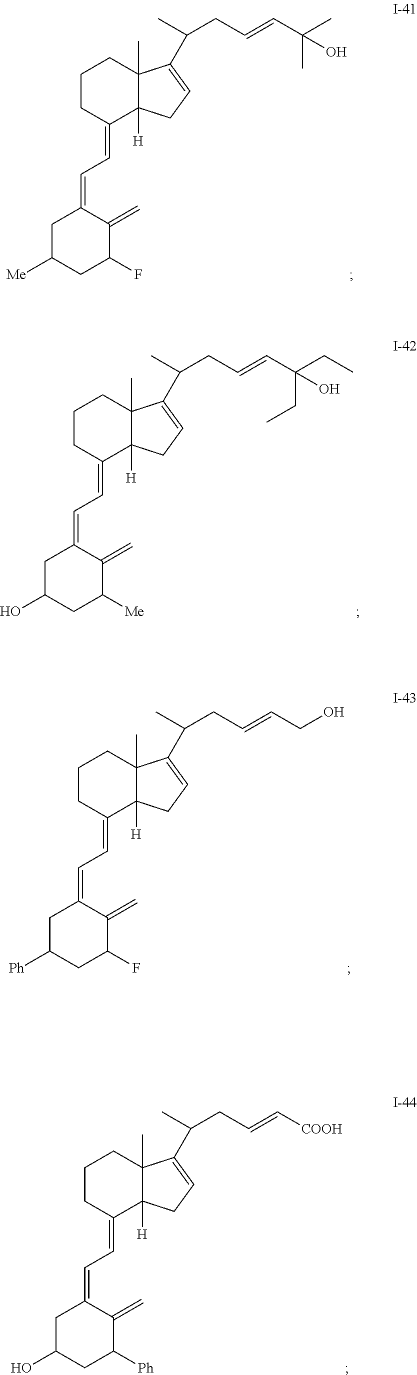 [SMILES: C=C1/C(=C\C=C2/CCCC3(C)C(C(C)C/C=C/C(C)(C)O)=CCC23)CC(C)CC1F ; C=C1/C(=C\C=C2/CCCC3(C)C(C(C)C/C=C/C(O)(CC)CC)=CCC23)CC(O)CC1C ; C=C1/C(=C\C=C2/CCCC3(C)C(C(C)C/C=C/CO)=CCC23)CC(c2ccccc2)CC1F ; C=C1/C(=C\C=C2/CCCC3(C)C(C(C)C/C=C/C(=O)O)=CCC23)CC(O)CC1c1ccccc1]

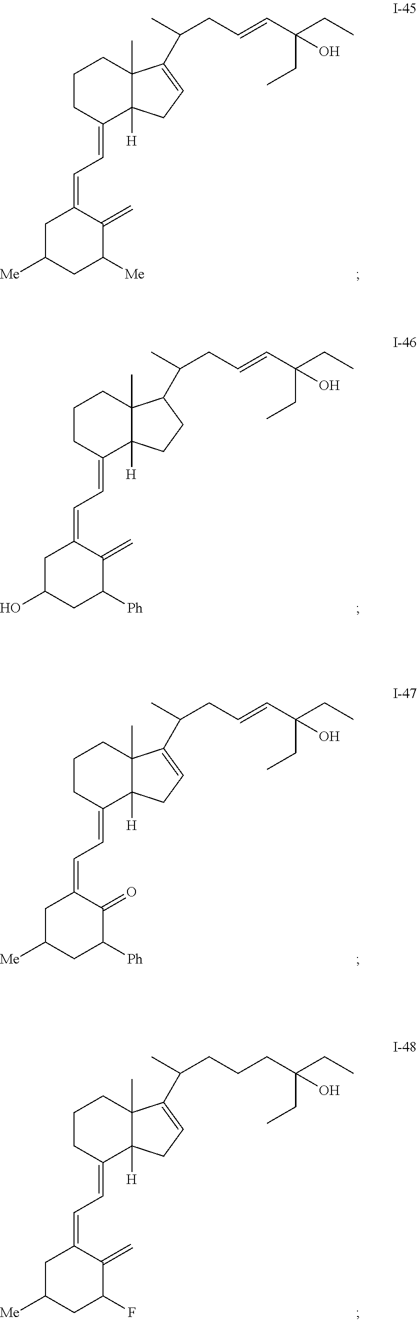 [SMILES: C=C1/C(=C\C=C2/CCCC3(C)C(C(C)C/C=C/C(O)(CC)CC)=CCC23)CC(C)CC1C ; C=C1/C(=C\C=C2/CCCC3(C)C2CCC3C(C)C/C=C/C(O)(CC)CC)CC(O)CC1c1ccccc1 ; CCC(O)(/C=C/CC(C)C1=CCC2/C(=C/C=C3/CC(C)CC(c4ccccc4)C3=O)CCCC12C)CC ; C=C1/C(=C\C=C2/CCCC3(C)C(C(C)CCCC(O)(CC)CC)=CCC23)CC(C)CC1F]

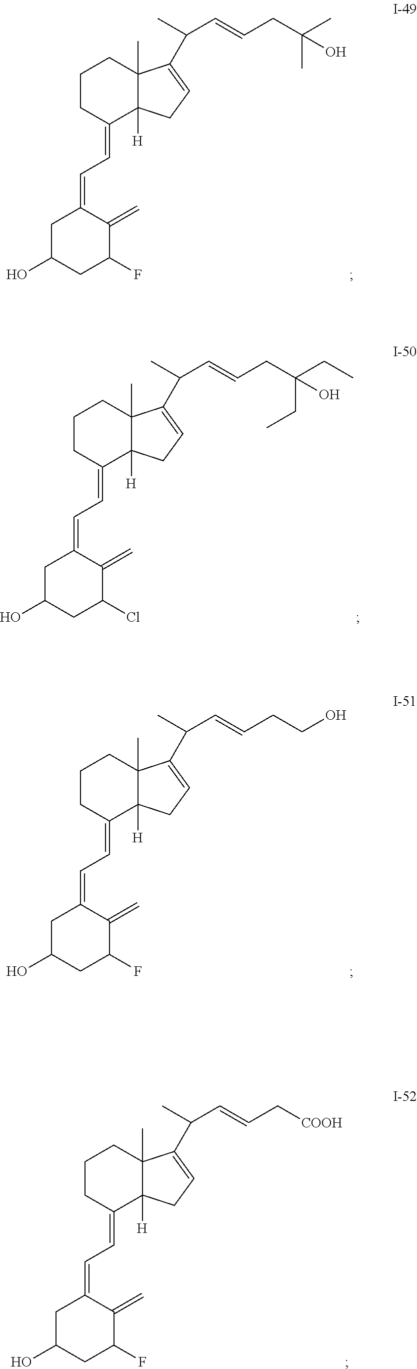 [SMILES: C=C1/C(=C\C=C2/CCCC3(C)C(C(C)/C=C/CC(C)(C)O)=CCC23)CC(O)CC1F ; C=C1/C(=C\C=C2/CCCC3(C)C(C(C)/C=C/CC(O)(CC)CC)=CCC23)CC(O)CC1Cl ; C=C1/C(=C\C=C2/CCCC3(C)C(C(C)/C=C/CCO)=CCC23)CC(O)CC1F ; C=C1/C(=C\C=C2/CCCC3(C)C(C(C)/C=C/CC(=O)O)=CCC23)CC(O)CC1F]

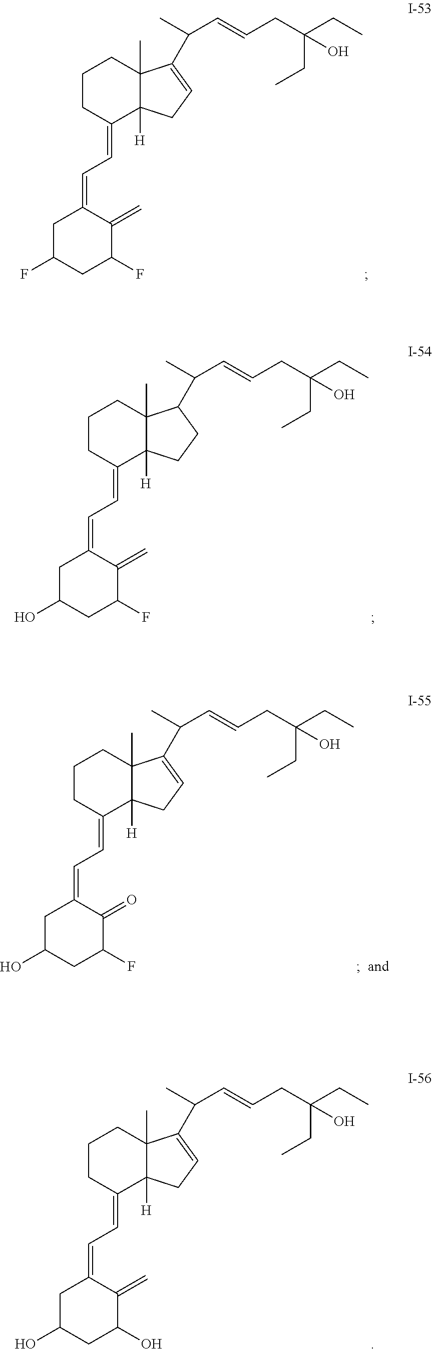 [SMILES: C=C1/C(=C\C=C2/CCCC3(C)C(C(C)/C=C/CC(O)(CC)CC)=CCC23)CC(F)CC1F ; C=C1/C(=C\C=C2/CCCC3(C)C2CCC3C(C)/C=C/CC(O)(CC)CC)CC(O)CC1F ; CCC(O)(CC)C/C=C/C(C)C1=CCC2/C(=C/C=C3/CC(O)CC(F)C3=O)CCCC12C ; C=C1/C(=C\C=C2/CCCC3(C)C(C(C)/C=C/CC(O)(CC)CC)=CCC23)CC(O)CC1O]

Further examples of vitamin D analogues suitable for use in the compositions and methods of the invention include calcitriol; paricalcitol (ZEMPLAR™) (see U.S. Pat. No. 5,587,497); tacalcitol (BONALFA™) (see U.S. Pat. No. 4,022,891); doxercalciferol (HECTOROL™) (see Lam et al. (1974) Science 186, 1038); maxacalcitol (OXAROL™) (see U.S. Pat. No. 4,891,364); calcipotriol (DAIVONEX™ (see U.S. Pat. No. 4,866,048); falecalcitriol (FULSTAN™); ecalcidene; calcithiazol; tisocalcitate; atocalcitol; lexacalcitol; seocalcitol; and secalciferol (“OSTEO D”). Other non-limiting examples of vitamin D analogues suitable for use in the invention include those described in published international applications: WO 01/40177, WO0010548, WO0061776, WO0064869, WO0064870, WO0066548, WO0104089, WO0116099, WO0130751, WO0140177, WO0151464, WO0156982, WO0162723, WO0174765, WO0174766, WO0179166, WO0190061, WO0192221, WO0196293, WO02066424, WO0212182, WO0214268, WO03004036, WO03027065, WO03055854, WO03088977, WO04037781, WO4067504, WO8000339, WO8500819, WO8505622, WO8602078, WO8604333, WO8700834, WO8910351, WO9009991, WO9009992, WO9010620, WO9100271, WO9100855, WO9109841, WO9112239, WO9112240, WO9115475, WO9203414, WO9309093, WO9319044, WO9401398, WO9407851, WO9407852, WO9408958, WO9410139, WO9414766, WO9502577, WO9503273, WO9512575, WO9527697, WO9616035, WO9616036, WO9622973, WO9711053, WO9720811, WO9737972, WO9746522, WO9818759, WO9824762, WO9828266, WO9841500, WO9841501, WO9849138, WO9851663, WO9851664, WO9851678, WO9903829, WO9912894, WO9915499, WO9918070, WO9943645, WO9952863, those described in: U.S. Pat. No. 3,856,780, U.S. Pat. No. 3,994,878, U.S. Pat. No. 4,021,423, U.S. Pat. No. 4,026,882, U.S. Pat. No. 4,028,349, U.S. Pat. No. 4,225,525, U.S. Pat. No. 4,613,594, U.S. Pat. No. 4,804,502, U.S. Pat. No. 4,898,855, U.S. Pat. No. 5,039,671, U.S. Pat. No. 5,087,619, U.S. Pat. No. 5,145,846, U.S. Pat. No. 5,247,123, U.S. Pat. No. 5,342,833, U.S. Pat. No. 5,428,029, U.S. Pat. No. 5,451,574, U.S. Pat. No. 5,612,328, U.S. Pat. No. 5,747,479, U.S. Pat. No. 5,804,574, U.S. Pat. No. 5,811,414, U.S. Pat. No. 5,856,317, U.S. Pat. No. 5,872,113, U.S. Pat. No. 5,888,994, U.S. Pat. No. 5,939,408, U.S. Pat. No. 5,962,707, U.S. Pat. No. 5,981,780, U.S. Pat. No. 6,017,908, U.S. Pat. No. 6,030,962, U.S. Pat. No. 6,040,461, U.S. Pat. No. 6,100,294, U.S. Pat. No. 6,121,312, U.S. Pat. No. 6,329,538, U.S. Pat. No. 6,331,642, U.S. Pat. No. 6,392,071, U.S. Pat. No. 6,452,028, U.S. Pat. No. 6,479,538, U.S. Pat. No. 6,492,353, U.S. Pat. No. 6,537,981, U.S. Pat. No. 6,544,969, U.S. Pat. No. 6,559,138, U.S. Pat. No. 6,667,298, U.S. Pat. No. 6,683,219, U.S. Pat. No. 6,696,431, U.S. Pat. No. 6,774,251, and those described in US Patent Publication Numbers: US20010007907, US20030083319, US20030125309, US20030130241, US20030171605, US20040167105. The foregoing patents and publications are incorporated by reference herein.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, urethane groups, and ester groups.

Non-limiting examples of alkyl groups include straight, branched, and cyclic alkyl groups. Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups.

Non-limiting examples of alkenyl groups include straight, branched, and cyclic alkenyl groups. The olefin or olefins of an alkenyl group can be, for example, E, Z, cis, trans, terminal, or exo-methylene.

Non-limiting examples of alkynyl groups include straight, branched, and cyclic alkynyl groups. The triple bond of an alkylnyl group can be internal or terminal.

A halo group can be any halogen atom, for example, fluorine, chlorine, bromine, or iodine.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl.

An aryloxy group can be, for example, an oxygen atom substituted with any aryl group, such as phenoxy.

An aralkyl group can be, for example, any alkyl group substituted with any aryl group, such as benzyl.

An arylalkoxy group can be, for example, an oxygen atom substituted with any aralkyl group, such as benzyloxy.

A heterocycle can be any ring containing a ring atom that is not carbon. A heterocycle can be substituted with any number of substituents, for example, alkyl groups and halogen atoms. A heterocycle can be aromatic or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

An acyl group can be, for example, a carbonyl group substituted with hydrocarbyl, alkyl, hydrocarbyloxy, alkoxy, aryl, aryloxy, aralkyl, arylalkoxy, or a heterocycle. Non-limiting examples of acyl include acetyl, benzoyl, benzyloxycarbonyl, phenoxycarbonyl, methoxycarbonyl, and ethoxycarbonyl.

An acyloxy group can be an oxygen atom substituted with an acyl group. An ester or an ester group comprises an acyloxy group. A non-limiting example of an acyloxy group, or an ester group, is acetate.

A carbamate group can be an oxygen atom substituted with a carbamoyl group, wherein the nitrogen atom of the carbamoyl group is unsubstituted, monosubstituted, or disubstituted with one or more of hydrocarbyl, alkyl, aryl, heterocyclyl, or aralkyl. When the nitrogen atom is disubstituted, the two substituents together with the nitrogen atom can form a heterocycle.

As used throughout, the structure: [symbol] , can be a single, double, or triple bond. In some embodiments, [symbol] is a single or double bond. In some embodiments, [symbol] is a single bond. In some embodiments, [symbol] is a double bond.

In some embodiments, at least one neurotherapeutic may be utilized in a pharmaceutical composition that contains at least one vitamin D analogue. A neurotherapeutic is defined herein as a chemical entity that may be used to prevent, reduce incidence of, treat, ameliorate the symptoms of, and/or diagnose a condition, disorder, or disease of the central and/or peripheral nervous system (herein collectively referred to as a “neurological disorder”) of a living organism.

In some embodiments, at least one cognitive enhancer neurotherapeutic may be utilized in a pharmaceutical composition that contains at least one vitamin D analogue. A cognitive enhancer is defined herein as a chemical entity that is used to improve a cognitive mental process. Non-limiting examples of a cognitive mental process include attention, memory, producing and understanding language, solving problems, and making decisions. A cognitive enhancer may come from one of many classes of drugs. Non-limiting examples of said drug-classes include cholinesterase inhibitors, glutamatergic molecules, N-methyl d-aspartate (NMDA) receptor antagonists, γ-Aminobutryic acid (GABA) receptor inverse agonists, GABA receptor antagonists, β-secretase inhibitors, α7-nicotinic receptor agonists, serotonin 5-HT₆ receptor antagonists, β1-adrenergic receptor agonists, and monoamine oxidase B (MAO-B) inhibitors.

In some embodiments, a cholinesterase inhibitor cognitive enhancer may be utilized in a pharmaceutical composition that contains at least one vitamin D analogue. In general, a cholinesterase inhibitor may reduce the rate at which cholinesterases digest neurotransmitters (e.g. acetylcholine) that are important to cognition, thereby allowing available neurotransmitter to pool in the brain. Non-limiting examples of cholinesterase inhibitors include donepezil, rivastigmine, galantamine, huperzine A, tacrine, dyflos, ecothiopate, Green mamba snake toxin fasciculin, metrifonate, heptyl-physostigmine, norpyridostigmine, norneostigmine, physostigmine, heptyl-physostigmine, velnacrine, citicoline, pyridostigmine, metrifonate, 7-methoxytacrine, eptastigmine, icopezil, ipidacrine, zifrosilone, anseculin, lactucopicrin, ungeremine, ladostigil, suronacrine, linopiridine, physostigmine, neostigmine, edrophonium, demacarium or ambenonium.

In some embodiments, a glutamatergic ampakine molecule cognitive enhancer may be utilized in a pharmaceutical composition. In general, ampakine molecules may bind strongly with the glutamatergic AMPA receptor and may improve cognition by increasing the concentration of the stimulatory neurotransmitter, glutamate, in the brain. Non-limiting examples of ampakines include 2-amino-3-(3-hydroxy-5-methyl-isoxazol-4-yl) propanoic acid (AMPA), aniracetam, piracetam, oxiracetam, 2-[2,6-difluoro-4-({2-[(phenylsulfonyl)amino]ethyl}thio)phenoxy]acetamide (PEPA), pramiracetam, sunifiram, unifiram, 6-(piperidin-1-ylcarbonyl)quinoxaline (CX-516), 2,3-dihydro-1,4-benzodioxin-7-yl-(1-piperidyl)methanone (CX-546), 2H,3H,6aH-pyrrolidino(2,1-3′,2′)1,3-oxazino(6′,5′-5,4)benzo(e)1,4-dioxan-10-one (CX-614), farampator, 7-chloro-3-methyl-3,4-dihydro-2H-1,2,4-benzothiadiazine 1,1-dioxide (IDRA-21), 4′-{(1S)-1-fluoro-2-[(isopropylsulfonyl)amino]-1-methylethyl}-N-methylbiphenyl-4-carboxamide (LY-503,430), N-[2-(4′-cyanobiphenyl-4-yl)propyl]propane-2-sulfonamide (LY-404,187), cyclothiazide, bendroflumethiazide, benzthiazide, buthiazide, chlorothiazide, epithiazide, hydrochlorothiazide, hydroflumethiazide, methylclothiazide, methalthiazide, polythiazide, or trichlormethiazdide.

In some embodiments, an NMDA-receptor antagonist cognitive enhancer may be utilized in a pharmaceutical composition. In general, an NMDA receptor antagonist may block the activity of the neurotransmitter glutamate to reduce nerve death caused by glutamate-linked excitotoxicity, and, thus, may improve cognition in subjects with late-stage impairment. Non-limiting examples of NMDA-receptor antagonists include memantine, amantadine, dextrallorphan, dextromethorphan, dextrorphan, dizocilpine, ethanol, eticyclidine, gacyclidine, ibogaine, magnesium, methoxetamine, nitrous oxide, phencyclidine, rolicyclidine, tenocyclidine, methoxydine, tiletamine, xenon, neramexane, eliprodil, etoxadrol, dexoxadrol, (2S,4S)-2-[(4S)-2,2-Diphenyl-1,3-dioxolan-4-yl]-4-fluoropiperidine (WMS-2539), (4aR,9aS)-N-Ethyl-4,4a,9,9a-tetrahydro-1H-fluoren-4a-amine (NEFA), remacemide delucemine, 8a-phenyldecahydroquinoline (8A-PDHQ), 1-aminocyclopropanecarboxylic acid (ACPC), 7-Chlorokynurenate, 5,7-dichlorokynuerenic acid (DCKA), kynurenic acid, lacosamide, aptiganel, dexanibinol, remacemide, rhynchophylline, ketamine, R-2-amino-5-phosphonopentanoate, 2-amino-7-phosphonoheptanoic acid, 3-[(R)-2-carboxypiperazin-4-yl]-prop-2-enyl-1-phosphonic acid, or selfotel.

In some embodiments, a GABA-receptor inverse agonist cognitive enhancer or GABA-receptor antagonist may be utilized in a pharmaceutical composition. An inverse agonist is defined herein as an agent that binds to the same receptor as a known agonist, yet the binding of said inverse agonist to said receptor generally results in a pharmacological response opposite in nature to that generated by said known agonist. In general, blocking (with an antagonist) or reversing (with an inverse agonist) the sedative action of the neurotransmitter γ-aminobutryic acid (GABA) at the GABA-receptor may improve cognition. In one non-limiting example, some benzodiazepine analogues may be inverse agonists for the GABA receptor. Other non-limiting examples of GABA-receptor inverse agonists include ethyl-8-azido-5,6-dihydro-5-methyl-6-oxo-4H-imidazo[1,4]-benzodiazepine-3-carboxylate (RO15-4513), methyl-6,7-dimethoxy-4-ethyl-beta-carboline-3-carboxylate (DMCM), methyl beta-carboline-3-carboxylate, ethyl beta-carboline-3-carboxylate, 3-methylaminocarbonyl-beta-carboline, methyl beta-carboline-3-carboxylate (β-CCM), 2-(3-isoxazolyl)-3,6,7,9-tetrahydroimidazo[4,5-d]pyrano+++[4,3-b] pyridine monophosphate monohydrate (S-8510), N-methyl-9H-pyrido[5,4-b]indole-3-carboxamide (FG-7142), ethyl 7-chloro-5-methyl-6-oxo-5,6-dihydro-4H-imidazo[1,5-a][1,4]benzodiazepine-3-carboxylate (RO 15-3505), 3,10-dichloro-9H-benzo[f]imidazo[1,5-a][1,2,4]triazolo[1,5-d][1,4]diazepine (R04882224), 3-bromo-10-difluoromethyl-9H-imidazo(1,5-a)(1,2,4)triazolo(1,5-d)(1,4)benzodiazepine (RO4938581), t-butyl 8-(trimethylsilyl) acetylene-5,6-dihydro-5-methyl-6-oxo-4H-imidazo[1,5a][1,4]benzodiazepine-3-carboxy-late (RY-023), ethyl-8-acetylene-5,6-dihydro-5-methyl-6-oxo-4H-imidazo[1,5a][1,4]benzodiazepine-3-carboxylate (RY-80), ethyl (S)-11,12,13,13a-tetrahydro-7-methoxy-9-oxo-9H-imidazo[1,5-a]pyrrolo[2,1-c][1,4]benzodiazepine-1-carboxylate (L-655,708), 3-(5-methylisoxazol-3-yl)-6-[(1-methyl-1H-1,2,3-triazol-4-yl)methoxy][1,2,4]triazolo[3,4-a]phthalazine (α₅IA), 8-chloro-3-(methoxymethyl)-5-methyl-4H-imidazo[1,5-a][1,4]benzodiazepin-6-one (PWZ-029), radequinil, terbequinil, or ethyl 5-isopropoxy-4-methyl-9H-β-carboline-3-carboxylate (ZK-93426).

Non-limiting examples of GABA-receptor antagonists include metrazol, bicucilline, gabazine, pitrazepin, quisqualamine, cicutoxin, cyclothiazide, flumazenil, oenanthotoxin, pentylenetetrazol, and picrotoxin.

In some embodiments, a β-secretase inhibitor cognitive enhancer may be utilized in a pharmaceutical composition that contains at least one Vitamin D analogue. In general, β-secretase inhibitors may be therapeutic in cases where cognitive impairment is due to the formation of endogenous plaques in the brain, such as amyloid-β protein plaques hypothesized in Alzheimer's disease. β-secretases have been implicated in plaque formation and inhibition of such enzymes may reduce the formation of said plaques, and, thus, improve cognition. Non-limiting examples of β-secretase inhibitors include P10-P4′ StatVal polypeptide (amino acid sequence: Lys-Thr-Glu-Glu-Ile-Ser-Glu-Val-Asn-Sta-Val-Ala-Glu-Phe, where Sta is a statin transition state mimetic), OM99-2 polypeptide (amino acid sequence: Glu-Val-Asn-Leu-Ala-Ala-Glu-Phe), KMI-429, KMI-570, KMI-684, 3-(1,1-dioxo-1λ {6},2-thiazinan-2-yl)-5-(ethylamino)-2-fluoro-N-[2S,3R)-3-hydroxy-1-phenyl-4-({[3-(trifluoromethyl)phenyl]methyl}amino)butan-2-yl]benzamide (GSK188909), N-(tert-Butoxycarbonyl)-L-leucine-N′-methoxy-N′-methylamide, and CTS21166.

In some embodiments, a α7-nicontinc receptor agonist cognitive enhancer may be utilized in a pharmaceutical composition that contains at least one vitamin D analogue. In generalαa7-nicontinc receptor agonists may be therapeutic as a large decrease in brain nicotinic receptors may be seen in some neurodegenerative diseases such as Alzheimer's disease. Such receptors are important activation points in pre- and post-synaptic excitation. Non-limiting examples of α7-nicontinc receptor agonists include EVP-6124, R3487, (+)-N-(1-azabicyclo[2.2.2]oct-3-yl)benzo[b] furan-2-carboxamide, A-582941, AR-R17779, TC-1698, TC-5619, GTS-21, PHA-543,613, PNU-282,987, PHA-709,829, SSR-180,711, tropisetron, WAY-317,538, anabaseine, choline, and nicotine.

In some embodiments, a serotonin 5-HT₆ receptor antagonist cognitive enhancer may be utilized in a pharmaceutical composition that contains at least one vitamin D analogue. In general, serotonin 5-HT₆ receptor antagonists may be therapeutic as, despite their functional excitatory action, 5-HT6 receptors are largely co-localized with GABAergic neurons and therefore produce an overall drop in brain activity. Reduction of such inactivity could improve cognition. Non-limiting examples of 5-HT₆ receptor antagonists include Lu AE58054, SYN120, BVT-5182, BVT-74316, cerlapiridine, EGIS-12233, latrepirdine, MS-245, PRX-07034, SB-271,046, SB-357,134, SB-399,885, SB-742,457, Ro04-6790, WAY-255315/SAM-315.

In some embodiments, a β1-adrenergic receptor agonist cognitive enhancer may be utilized in a pharmaceutical composition that contains at least one vitamin D analogue. In general, β1-adrenergic receptor agonists may be therapeutic as they can provide support to deteriorating noradrenergic afferents that extend from the locus coeruleus to the hippocampus. Non-limiting examples of β1-adrenergic receptor agonists include epinephrine, isoproterenol, dobutamine, and xamoterol.

In some embodiments, a MAO-B inhibitor cognitive enhancer may be utilized in a pharmaceutical composition that contains at least one vitamin D analogue. MAO-B aids in the degradation of the neurotransmitter dopamine in the brain and also contributes to free-radical formation that can lead to oxidative stress. Oxidative stress has been linked to neurodegenerative diseases, including Alzheimer's disease. In general, MAO-B inhibitors may be therapeutic as inhibitors of MAO-B may reduce subsequent free radical formation and, thus, oxidative stress than may be a factor in the development of a neurological disorder. Non-limiting examples of MAO-B inhibitors include RO4602522, EVT 302, geiparvarin, desmethoxyyangonin, catechin, epicatechin, harmala alkaloids, safinamide, 2-(N-Methyl-N-benzylaminomethyl)-1H-pyrrole, 1-(4-Arylthiazol-2-yl)-2-(3-methylcyclohexylidene) hydrazine, 2-Thiazolylhydrazone, 3,5-Diaryl pyrazole, coumarins, phenylcoumarins, chromone-3-phenylcarboxamides, isatins, phthalimides, 8-Benzyloxycaffeines, (E,E)-8-(4-phenylbutadien-1-yl)caffeines, selegiline, and rasagiline.

In some embodiments, a neurotherapeutic that may be used to treat multiple sclerosis may be utilized in a pharmaceutical composition. Non-limiting examples of neurotherapeutics used to treat multiple sclerosis include interferon β-1a, interferon β-1b, glatiramer, fingolimod, natalizumab, mitoxantrone, or teriflunomide.

In some embodiments, a neurotherapeutic that may be used to treat Parkinson's disease may be utilized in a pharmaceutical composition. Non-limiting examples of neurotherapeutics used to treat Parkinson's disease include levodopa, carbidopa, pramipexole, ropinirole, apomorphine, selegiline, rasagiline, benztropine, amantadine, bromocriptine, rotigotine, trihexyphenidyl, entacapone, or tolcapone.

In some embodiments, an anti-convulsant neurotherapeutic may be utilized in a pharmaceutical composition. Non-limiting examples of anti-convulsant neurotherapeutics include felbamate, gabapentin, lamotrigine, topiramate, tiagabine, diazepam, pregalbin, phenytoin, phenobarbital, carbamazepine, oxcarbazepine, vigabatrin, valproic acid, zonisamide, levetiracetam, clonazepam, rufinamide, acetazolamide, ethosuximide, primidone, clobazam, fosphenytoin, divalproex, ezogabine, or trimethadione.

In some embodiments, a neurotherapeutic used to treat neuropathic pain may be utilized in a pharmaceutical composition. Non-limiting examples of a neurotherapeutic used to treat neuropathic pain include anti-convulsants, an anti-depressant (e.g. selective serotonin reuptake inhibitors, amitriptyline, nortriptiline), steroids (e.g. methylprednisolone), glutamate antagonists, cytokine inhibitors, vaniloid-receptor agonists, catecholamine modulators, ion-channel blockers, opioids, cannabinoids, COX inhibitors (e.g. celecoxib, rofecoxib, valdecoxib, parecoxib, lumiracoxib, etoricoxib, firocoxib), acetylcholine modulators, adenosine receptor agonists, or non-steroidal anti-inflammatory drugs (e.g. ibuprofen, naproxen, acetaminophen, aspirin, acetaminophen, piroxicam, indomethacin).

The invention provides pharmaceutically-acceptable salts of any chemical entity described herein. Pharmaceutically-acceptable salts include, for example, acid-addition salts and base-addition salts. The acid that is added to a compound to form an acid-addition salt can be an organic acid or an inorganic acid. A base that is added to a compound to form a base-addition salt can be an organic base or an inorganic base. In some embodiments, a pharmaceutically-acceptable salt is a metal salt. In some embodiments, a pharmaceutically-acceptable salt is an ammonium salt.

Acid addition salts can arise from the addition of an acid to a compound described herein. In some embodiments, the acid is organic. In some embodiments, the acid is inorganic. Non-limiting examples of suitable acids include hydrochloric acid, hydrobromic acid, hydroiodic acid, nitric acid, nitrous acid, sulfuric acid, sulfurous acid, a phosphoric acid, nicotinic acid, isonicotinic acid, lactic acid, salicylic acid, 4-aminosalicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, citric acid, oxalic acid, maleic acid, hydroxymaleic acid, methylmaleic acid, glycolic acid, malic acid, cinnamic acid, mandelic acid, 2-phenoxybenzoic acid, 2-acetoxybenzoic acid, embonic acid, phenylacetic acid, N-cyclohexylsulfamic acid, methanesulfonic acid, ethanesulfonic acid, benzenesulfonic acid, p-toluenesulfonic acid, 2-hydroxyethanesulfonic acid, ethane-1,2-disulfonic acid, 4-methylbenzenesulfonic acid, naphthalene-2-sulfonic acid, naphthalene-1,5-disulfonic acid, 2-phosphoglyceric acid, 3-phosphoglyceric acid, glucose-6-phosphoric acid, and an amino acid.

Non-limiting examples of suitable acid addition salts include a hydrochloride salt, a hydrobromide salt, a hydroiodide salt, a nitrate salt, a nitrite salt, a sulfate salt, a sulfite salt, a phosphate salt, a hydrogen phosphate salt, a dihydrogen phosphate salt, a carbonate salt, a bicarbonate salt, a nicotinate salt, an isonicotinate salt, a lactate salt, a salicylate salt, a 4-aminosalicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a citrate salt, an oxalate salt, a maleate salt, a hydroxymaleate salt, a methylmaleate salt, a glycolate salt, a malate salt, a cinnamate salt, a mandelate salt, a 2-phenoxybenzoate salt, a 2-acetoxybenzoate salt, an embonate salt, a phenylacetate salt, an N-cyclohexylsulfamate salt, a methanesulfonate salt, an ethanesulfonate salt, a benzenesulfonate salt, a p-toluenesulfonate salt, a 2-hydroxyethanesulfonate salt, an ethane-1,2-disulfonate salt, a 4-methylbenzenesulfonate salt, a naphthalene-2-sulfonate salt, a naphthalene-1,5-disulfonate salt, a 2-phosphoglycerate salt, a 3-phosphoglycerate salt, a glucose-6-phosphate salt, and an amino acid salt.

Metal salts can arise from the addition of an inorganic base to a compound described herein. Non-limiting examples of suitable metals include lithium, sodium, potassium, cesium, cerium, magnesium, manganese, iron, calcium, strontium, cobalt, titanium, aluminum, copper, cadmium, and zinc. Non-limiting examples of suitable metal salts include a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, and a zinc salt.

Non-limiting examples of suitable ammonium salts include a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzyl amine salt, a piperazine salt, a pyridine salt, a pyrrazole salt, a pipyrrazole salt, an imidazole salt, a pyrazine salt, a pipyrazine salt, an ethylene diamine salt, an N,N′-dibenzylethylene diamine salt, a procaine salt, a chloroprocaine salt, a choline salt, a dicyclohexyl amine salt, and a N-methylglucamine salt.

Methods of the Invention

The methods of the invention can be administered, guided, and modified based on a personalized medicine approach. Personalized medicine provides health care methods adapted to the needs of a specific subject as opposed to methods established over medical cohorts and epidemiological studies, subsequently applied to an individual. Personalized medicine allows a health care provider to optimize a therapy for a specific subject based on a number of factors, for example, genetics, metabolism, family history, personal history, environment, behavior, diet, lifestyle, social tendencies, and personal goals. At any time before or during the therapy, a health care provider can investigate any relevant factor and use the resulting information to design or improve a therapeutic regimen. Investigation can include an assay described herein or personal counseling between the health care provider and the subject. Personalized medicine allows for a therapy to be combined with companion diagnostic tests to select a sub-population of patients who would benefit from the treatments described herein.

In some embodiments, the invention provides a method of reducing incidence of or treating a neurological disorder, the method comprising administering a therapeutically effective amount of a vitamin D analogue, or a pharmaceutically-acceptable salt thereof, to a subject in need or want thereof.

In some embodiments, the invention provides a method of reducing incidence of or treating a neurological disorder, the method comprising administering a therapeutically effective amount of a Vitamin D analogue, elocalcitol, or a pharmaceutically-acceptable salt thereof, to a subject in need or want thereof.

In some embodiments, the invention provides a method of reducing incidence of or treating a neurological disorder, the method comprising administering a therapeutically effective amount of a vitamin D analogue, or a pharmaceutically-acceptable salt thereof, and a therapeutically effective amount of a neurotherapeutic, or a pharmaceutically-acceptable salt thereof, to a subject in need or want thereof.

In some embodiments, the invention provides a method of reducing incidence of or treating a neurological disorder, the method comprising administering a therapeutically effective amount of a Vitamin D analogue, elocalcitol, or a pharmaceutically-acceptable salt thereof, and a therapeutically effective amount of a neurotherapeutic, or a pharmaceutically-acceptable salt thereof, to a subject in need or want thereof.

In some embodiments, the invention provides a method wherein a vitamin D analogue and at least one neurotherapeutic may be administered separately (e.g. administered at different times).

In some embodiments, the invention provides a method wherein a vitamin D analogue and at least one neurotherapeutic may be administered simultaneously (e.g. administered at the same time).

In some embodiments, the invention provides a method wherein elocalcitol and at least one neurotherapeutic may be administered together in a single formulation.

In some embodiments, the invention provides a method wherein a vitamin D analogue and at least one neurotherapeutic may be may be synergistic agents and either or both of said vitamin D analogue and at least one said neurotherapeutic have lower therapeutically effective doses when administered in combination than when each agent is administered in the absence of the other.

In some embodiments, the invention provides compounds for reducing incidence of or treating a neurological disorder, the method comprising administering a therapeutically effective amount of a vitamin D analogue, or a pharmaceutically-acceptable salt thereof, to a subject in need or want thereof.

In some embodiments, the invention provides compounds for reducing incidence of or treating a neurological disorder, the method comprising administering a therapeutically effective amount of the vitamin D analogue, elocalcitol, or a pharmaceutically-acceptable salt thereof, to a subject in need or want thereof.

In some embodiments, the invention provides compounds for reducing incidence of or treating a neurological disorder, the method comprising administering a therapeutically effective amount of a vitamin D analogue, or a pharmaceutically-acceptable salt thereof, and a therapeutically effective amount of a neurotherapeutic, or a pharmaceutically-acceptable salt thereof, to a subject in need or want thereof.

In some embodiments, the invention provides compounds for reducing incidence of or treating a neurological disorder, the method comprising administering a therapeutically effective amount of a vitamin D analogue, elocalcitol, or a pharmaceutically-acceptable salt thereof, and a therapeutically effective amount of a neurotherapeutic, or a pharmaceutically-acceptable salt thereof, to a subject in need or want thereof.

In some embodiments, the invention provides compounds for the preparation of a medicament for reducing incidence of or treating a neurological disorder, the method comprising administering a therapeutically effective amount of a vitamin D analogue, or a pharmaceutically-acceptable salt thereof, to a subject in need or want thereof.

In some embodiments, the invention provides compounds for the preparation of a medicament for reducing incidence of or treating a neurological disorder, the method comprising administering a therapeutically effective amount of the vitamin D analogue, elocalcitol, or a pharmaceutically-acceptable salt thereof, a subject in need or want thereof.

In some embodiments, the invention provides compounds for the preparation of a medicament for reducing incidence of or treating a neurological disorder, the method comprising administering a therapeutically effective amount of a vitamin D analogue, or a pharmaceutically-acceptable salt thereof, and a therapeutically effective amount of a neurotherapeutic, or a pharmaceutically-acceptable salt thereof, to a subject in need or want thereof.

In some embodiments, the invention provides compounds for the preparation of a medicament for reducing incidence of or treating a neurological disorder, the method comprising administering a therapeutically effective amount of a vitamin D analogue, elocalcitol, or a pharmaceutically-acceptable salt thereof, and a therapeutically effective amount of a neurotherapeutic, or a pharmaceutically-acceptable salt thereof, to a subject in need or want thereof.

A subject for any of the therapeutic methods disclosed herein can be a subject in need or want of therapy for one or more conditions disclosed herein. The subject can be a human. Other non-limiting examples of a subject include non-human animals, such as companion animals, pets, livestock, service animals, guardian animals, laboring animals, and zoo animals. In some embodiments, the animal is a mammal.

Non-limiting examples of a neurological disorder that may be treated or the incidence of reduced by methods and compositions of the invention include neuropathic pain, genetically-linked disorders (e.g. Huntington's disease, muscular dystrophy, Charcot-Marie-Tooth neuropathy), developmental disorders (e.g. spina bifida), degenerative disorders (e.g. Alzheimer's disease, Parkinson's disease, amyotrophic lateral sclerosis, dementia), disorders associated with blood flow in, to, or from the brain (e.g. stroke), injury-related disorders (e.g. spinal cord damage, brain damage, concussion), seizure disorders (e.g. epilepsy), infection disorders (e.g. meningitis), autoimmune disorders (e.g. multiple sclerosis, Guillain-Barré Syndrome), episodic disorders (e.g. migraines), or cancer (e.g. brain tumors).

In some embodiments, the invention provides methods for reducing incidence of a neurological disorder in a subject. In some embodiments, a subject may be identified as at-risk for a given neurological disorder based on, for example, a genetic predisposition to the neurological disorder (e.g., having a genetic mutation associated with the neurological disorder, having a genotype associated with the neurological disorder), familial history of the neurological disorder, age, gender, blood chemistry, having another condition associated with the neurological disorder, environmental factors (e.g., exposure to agents known to cause the neurological condition), any other characteristic associated with the neurological disorder, and combinations thereof.

To reduce incidence of the neurological disorder in the at-risk subject, the at-risk subject may be administered a therapeutically effective amount of a vitamin D analogue (e.g., including any vitamin D analogue provided herein), or a pharmaceutically-acceptable salt thereof; a therapeutically effective amount of a neurotherapeutic, or a pharmaceutically-acceptable salt thereof; or a combination thereof. Treatment of the subject with the vitamin D analogue and/or neurotherapeutic may continue over a fixed period of time or indefinitely. In some embodiments, the vitamin D analogue is elocalcitol. In some embodiments, the therapeutically effective amount of the vitamin D analogue is one that does not exceed a threshold over which hypercalcemia is induced.

In some embodiments, the invention provides methods for reducing incidence of Parkinson's disease in a subject. In some embodiments, a subject may be identified as at-risk for developing Parkinson's disease and treated with either or both of a vitamin D analogue (e.g., elocalcitol or any other vitamin D analogue provided herein) and a neurotherapeutic, including, for example, those described for Parkinson's disease herein. The subject may be identified as at-risk for Parkinson's disease, for example, as having a familial history of Parkinson's disease, exposure to environmental factors associated with Parkinson's disease, and/or possessing one or more genetic predispositions to Parkinson's disease. Non-limiting examples of genetic predispositions to Parkinson's disease include genetic mutations in genes that code for alpha-synuclein (SNCA, encoded by the SNCA gene), parkin (PRKN, encoded by the PARK2 gene), leucine-rich repeat kinase 2 (LRRK2, coded by the PARK8 gene (also known as Dardarin)), PTEN-induced putative kinase 1 (PINK1, encoded by the PINK1 gene), DJ-1 (encoded by the PARK7 gene), ATP-13A2 (encoded by the ATP13A2 gene), and glucocerebrosidase (GBA, encoded by the GBA gene). Treatment of the at-risk subject with the vitamin D analogue and/or neurotherapeutic may continue over a fixed period of time or may continue indefinitely.

In some embodiments, the invention provides methods for reducing incidence of Alzheimer's disease in a subject. In some embodiments, a subject may be identified as at-risk for developing Alzheimer's disease and treated with either or both of a vitamin D analogue (e.g., elocalcitol or any other vitamin D analogue described herein) and a neurotherapeutic, including, for example, cognitive enhancers and others provided for Alzheimer's disease herein. The subject may be identified as at-risk for Alzheimer's disease, for example, as having a familial history of Alzheimer's disease, exposure to environmental factors associated with Alzheimer's disease, possessing a condition associated with Alzheimer's disease (e.g., reduced production of the neurotransmitter acetylcholine, infection with Herpes simplex virus-1, age-related myelin breakdown, loss of locus coeruleus cells that provide norepinephrine, tau protein abnormalities, oxidative stress and dys-homeostasis of biometal metabolism), and/or possessing one or more genetic predispositions to Alzheimer's disease. Non-limiting examples of genetic predispositions to Alzheimer's disease include genetic mutations in genes that code for amyloid precursor protein (AP), presenilins 1 and 2, and TREM2; and possession of at least one APOEε4 allele. Treatment of the at-risk subject with the vitamin D analogue and/or neurotherapeutic may continue over a fixed period of time or may continue indefinitely.

Pharmaceutical Compositions of the Invention

A pharmaceutical composition of the invention can comprise one or more vitamin D analogues. The compounds can be present in any ratio and can be administered, for example, by any method described herein or otherwise known to one of skill in the art. A pharmaceutical composition optionally further comprises any number of pharmaceutically-acceptable excipients, additives, or vehicles.

A pharmaceutical composition of the invention can comprise one or more vitamin D analogues and one or more neurotherapeutics. The compounds can be present in any ratio and can be administered, for example, by any method described herein or otherwise known to one of skill in the art. A pharmaceutical composition optionally further comprises any number of pharmaceutically-acceptable excipients, additives, or vehicles.

A pharmaceutical composition of the invention can be administered to a subject along with pharmaceutical excipients or diluents. These compositions can take the form of drops, solutions, suspensions, tablets, pills, capsules, powders, sustained-, controlled-, or instant-release formulations, and other formulations known in the art. A pharmaceutical composition of the invention could be modulated using suitable excipients and diluents.

A pharmaceutical composition of the invention can be formulated in a unit dosage form, each dosage containing, for example, from about 0.01 mg to 1000 mg of a vitamin D analogue. In some embodiments, a dose contains less than 1000, 500, 300, 200, 100, 50, 25, 10, 5, 1, 0.1, or 0.01 mg of a vitamin D analogue. In some embodiments, a dose contains 0.01 mg to 1000 mg of elocalcitol. In some embodiments, a dose contains less than 1000, 500, 300, 200, 100, 50, 25, 10, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.005, or 0.001 mg elocalcitol. In some embodiments, a dose contains 75 to 150 μg. In some embodiments, a dose contains, from about 0.01 mg to 1000 mg of a neurotherapeutic. In some embodiments, a dose contains less than 1000, 500, 300, 200, 100, 50, 25, 10, 5, 1, 0.1, or 0.01 mg of a neurotherapeutic. In some embodiments, elocalcitol monotherapy or elocalcitol/neurotherapeutic combination therapy may be administered for at least two weeks. In some embodiments, elocalcitol monotherapy or elocalcitol/neurotherapeutic combination therapy may be administered for at least four weeks. In some embodiments, elocalcitol monotherapy or elocalcitol/neurotherapeutic combination therapy may be administered for at least three months, six months, twelve months, 2 years, 3 years, 4 years, 5 years, 10 years, 15 years, 20 years, 25 years, 30 years, or 40 years. In some embodiments, elocalcitol monotherapy or elocalcitol/neurotherapeutic combination therapy may be administered to a subject in want or need, for the duration of the subject's lifetime.

A unit dosage form can be administered to humans, domestic pets, livestock, or other animals with a pharmaceutically-acceptable diluents or excipients. Administration can be topical, intraaural, parenteral, intravenous, intraarterial, subcutaneous, intramuscular, intracranial, intraorbital, ophthalmic, intraventricular, intracapsular, intraspinal, intracisternal, intraperitoneal, intranasal, aerosol, oral, or by suppository.

The dosage of pharmaceutical compositions of the present invention can vary depending on the symptoms, age and body weight of the subject, the nature and severity of the disorder to be treated, the route of administration, and the form of the composition. Any pharmaceutical composition of the invention can be administered in a single dose or in divided doses. Dosages can be readily determined by techniques known to those of skill in the art or as taught herein. In some embodiments, the dosage of a vitamin D analogue may be in the range of about 0.01 ng to about 10 g per kg of the subject's body mass. In some embodiments the dosage of a vitamin D analogue may be in the range of about 1 ng to about 0.1 g per kg. In some embodiments, the dosage of a vitamin D analogue may be in the range of about 100 ng to about 10 mg per kg. In some embodiments, the dosage of a vitamin D analogue may be from about 75 μg to 150 μg per day.

In some embodiments, the dosage of a neurotherapeutic may be in the range of about 0.01 ng to about 10 g per kg of the subject's body mass. In some embodiments the dosage of a neurotherapeutic may be in the range of about 1 ng to about 0.1 g per kg. In some embodiments, the dosage of a neurotherapeutic may be in the range of about 100 ng to about 10 mg per kg.

In some embodiments, the dosage of elocalcitol may be in the range of about 0.01 ng to about 10 g per kg of the subject's body mass. In some embodiments the dosage of elocalcitol may be in the range of about 1 ng to about 0.1 g per kg. In some embodiments, the dosage of elocalcitol may be in the range of about 100 ng to about 10 mg per kg.

An exemplary dose range for a vitamin D analogue is from 0.1 to 300 μg per day, for example, 50-150 μg per day, or 75 or 150 μg per day. A unit dose formulation can contain 50-150 μg e.g., 75 μg or 150 μg, and can be administered once per day. Another exemplary dose is one that is administered at a concentration of about 0.001 μg to about 100 μg per kilogram of the subject's body mass, about 0.001 to about 10 μg /kg, or about 0.001 μg to about 100 μg kg of body mass. Ranges intermediate to the above-recited values are also intended to be part of the invention.

An exemplary dose range for elocalcitol is from about 0.1 to 300 μg per day, for example, 50-150 μg per day, or 75 to 150 μg per day. A unit dose formulation can contain 50-150 μg e.g., 75 μg or 150 μg, and can be administered once per day. Another exemplary dose is one that is administered at a concentration of about 0.001 μg to about 100 μg per kilogram of the subject's body mass, about 0.001 to about 10 μg /kg, or about 0.001 μg to about 100 μg/kg of body mass. Ranges intermediate to the above-recited values are also intended to be part of the invention.

The combined use of multiple compounds in a pharmaceutical composition of the present invention can reduce the required dosage for any individual compound. In such combined therapy, the compounds can be delivered together or separately, simultaneously or at different times.

The pharmaceutical compositions of the present invention can be administered by various means known in the art. For oral administration, a pharmaceutical composition of the invention can be formulated as tablets, capsules, granules, powders, or syrups. Pharmaceutical compositions of the present invention can be administered parenterally as injections (intravenous, intramuscular or subcutaneous), drop infusion preparations, or suppositories.

### EXAMPLES

### Example 1

A subject is experiencing symptoms of Alzheimer's disease and is diagnosed with the disease. The subject may be prescribed a monotherapy of a vitamin D analogue wherein the vitamin D analogue is elocalcitol. Elocalcitol is prescribed at a dosage of 100 μg daily and administered in a once daily oral formulation. Alternatively, the subject may be prescribed a combination therapy of elocalcitol, and the neurotherapeutic directed toward Alzheimer's disease, donzepil. Elocalcitol is prescribed at a dosage of 100 μg daily and donzepil at a dosage of 5 mg daily. Combination therapy is administered together in a single formulation comprising both elocalcitol and donzepil and given once daily. In both the cases of monotherapy and combination therapy, therapy is continuous for at least six months. The subject is monitored throughout the course of therapy to determine therapeutic efficacy, minimized side-effects (e.g. hypercalcemia), and any need for changes to dosing or the prescribed therapeutic regimen.

### Example 2

A subject is experiencing symptoms of multiple sclerosis and is diagnosed with the disease. The subject may be prescribed a monotherapy of a vitamin D analogue wherein the vitamin D analogue is elocalcitol. Elocalcitol is prescribed at a dosage of 125 μg daily and administered in a once daily oral formulation. Alternatively, the subject may be prescribed a combination therapy of elocalcitol, and the neurotherapeutic directed toward multiple sclerosis, glatiramer. Elocalcitol is prescribed at a dosage of 125 μg daily and galtiramer at a dosage of 20 mg per day. Elocalcitol and glatiramer are administered separately, elocalcitol in an oral formulation and glatiramer as a subcutaneous injection, each given once per day. In both the cases of monotherapy and combination therapy, therapy is continuous indefinitely. Moreover, the subject is monitored throughout the course of therapy to determine therapeutic efficacy, minimized side-effects (e.g. hypercalcemia), and any need for changes to dosing or the prescribed therapeutic regimen.

### Example 3

A subject is experiencing symptoms of Parkinson's disease and is diagnosed with the disease. The subject may be prescribed a monotherapy of a vitamin D analogue wherein the vitamin D analogue is elocalcitol. Elocalcitol is prescribed at a dosage of 135 μg daily and administered in a once daily oral formulation. Alternatively, the subject may be prescribed a combination therapy of elocalcitol, and the neurotherapeutics directed toward Parkinson's disease, levodopa and carbidopa. Elocalcitol is prescribed at a dosage of 135 μg daily, levodopa at 300 mg daily, and carbidopa at 75 mg daily. Elocalcitol is administered in an oral dosage form once daily and levodopa and carbidopa are administered together in a separate oral dosage form. As levidopa and carbidopa are generally given several times a day, the daily doses of levodopa and carbidopa shown above may be divided into several more frequent (e.g. greater than once per day) doses that, when combined, do not exceed the daily doses shown above. In both the cases of monotherapy and combination therapy, therapy is continuous for at least six months. Moreover, the subject is monitored throughout the course of therapy to determine therapeutic efficacy, minimized side-effects (e.g. hypercalcemia), and any need for changes to dosing or the prescribed therapeutic regimen.

### Example 4

A subject is experiencing symptoms of epilepsy disease and is diagnosed with the disease. The subject may be prescribed a monotherapy of a vitamin D analogue wherein the vitamin D analogue is elocalcitol. Elocalcitol is prescribed at a dosage of 75 μg daily and administered in an oral formulation once daily. Alternatively, the subject may be prescribed a combination therapy of elocalcitol, and the neurotherapeutic directed toward epilepsy, gabapentin. Elocalcitol is prescribed at a dosage of 75 μg daily and gabapentin at 900 mg daily. Combination therapy is administered separately, with elocalcitol given in one oral formulation once daily. The daily dose of gabapentin is divided over several doses per day that, when combined, do not exceed the daily dose shown above. In both the cases of monotherapy and combination therapy, therapy is continuous for at least 90 days. Moreover, the subject is monitored throughout the course of therapy to determine therapeutic efficacy, minimized side-effects (e.g. hypercalcemia), and any need for changes to dosing or the prescribed therapeutic regimen.

### Example 5

A subject is experiencing symptoms of chronic neuropathic pain. The subject may be prescribed a monotherapy of a vitamin D analogue wherein the vitamin D analogue is elocalcitol. Elocalcitol is prescribed at a dosage of 150 μg daily administered as an oral formulation given once daily. Alternatively, the subject may be prescribed a combination therapy of elocalcitol, and the neurotherapeutic directed toward neuropathic pain, naproxen. Elocalcitol is prescribed at a dosage of 150 μg daily and naproxen at 500-1000 mg daily. Combination therapy is administered together in one oral formulation and given twice daily, with elocalcitol and naproxen doses administered at half the daily dose in each bi-daily dose. In both the cases of monotherapy and combination therapy, therapy is continuous for at least 30 days. Moreover, the subject is monitored throughout the course of therapy to determine therapeutic efficacy, minimized side-effects (e.g. hypercalcemia), and any need for changes to dosing or the prescribed therapeutic regimen.

### Example 6

A subject is identified as at-risk for Alzheimer's disease as possessing at least one APOEε4 allele. To reduce incidence of Alzheimer's disease in the subject, the subject may be prescribed a monotherapy of a vitamin D analogue, wherein the vitamin D analogue is elocalcitol. Elocalcitol is prescribed at a dosage of 100 μg daily and administered in a once daily oral formulation. Therapy is continuous for at least six months and may continue indefinitely. Moreover, the subject is monitored throughout the course of therapy to determine therapeutic efficacy (e.g., the ability of the therapeutic regimen to reduce incidence of Alzheimer's disease in the subject), minimized side-effects (e.g. hypercalcemia), and any need for changes to dosing or the prescribed therapeutic regimen.

### Example 7

A subject is identified as at-risk for Parkinson's disease as possessing one or more

Parkinson's relevant gene mutations in the PARK8 gene that encodes for LRRK2. To reduce incidence of Parkinson's disease in the subject, the subject may be prescribed a monotherapy of a vitamin D analogue, wherein the vitamin D analogue is elocalcitol. Elocalcitol is prescribed at a dosage of 135 μg daily and administered in a once daily oral formulation. Therapy is continuous for at least six months and may continue indefinitely. Moreover, the subject is monitored throughout the course of therapy to determine therapeutic efficacy (e.g., the ability of the therapeutic regimen to reduce incidence of Parkinson's disease in the subject), minimized side-effects (e.g. hypercalcemia), and any need for changes to dosing or the prescribed therapeutic regimen.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EMBODIMENTS

The following embodiments provide non-limiting examples of embodiments of the invention.

### Embodiment 1

A pharmaceutical composition comprising: a) a vitamin D analogue, or a pharmaceutically-acceptable salt thereof; b) a neurotherapeutic, or a pharmaceutically-acceptable salt thereof; and c) optionally, a pharmaceutically-acceptable excipient.

### Embodiment 2

The pharmaceutical composition of embodiment 1, wherein the vitamin D analogue is a vitamin D analogue of the formula:

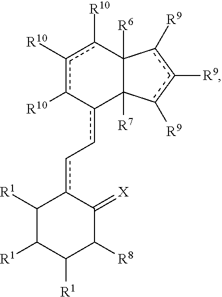

wherein:
each R¹, R⁶, R⁷, R⁸, R⁹ and R¹⁰ is independently H, halo, alkyl, alkenyl, alkynyl, aryl, aralkyl, hydroxyl, sulfhydryl, amino, nitro, cyano, alkoxy, an ester group, an amide group, a carbonate group, a carbamate group, or is a group of the formula:

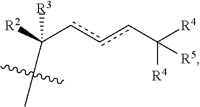

wherein:
each R², R³, R⁴, and R⁵ is independently H, halo, alkyl, aryl, or hydroxyl, X is C(R¹)₂, 0, or NR¹; and each alkyl, alkenyl, alkynyl, aryl, aralkyl, amino, and alkoxy group is optionally substituted.

### Embodiment 3

The pharmaceutical composition of embodiment 1, wherein the vitamin D analogue is a vitamin D analogue of the formula:

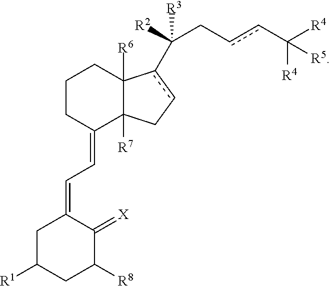

### Embodiment 4

The pharmaceutical composition of embodiment 3, wherein: R¹ is H, halo, alkyl, or hydroxyl; R² is H or alkyl; R³ is H or alkyl; each R⁴ is independently H or alkyl; R⁵ is H, alkyl, or hydroxyl; R⁶ is H or alkyl; R⁷ is H or alkyl; R⁸ is H, halo, alkyl, or hydroxyl; and X is CH₂ or O.

### Embodiment 5

The pharmaceutical composition of embodiment 1, wherein the vitamin D analogue is any one of compounds (I-01)-(I-56).

### Embodiment 6

The pharmaceutical composition of embodiment 1, wherein the vitamin D analogue is elocalcitol.

### Embodiment 7

The pharmaceutical composition of any one of embodiments 1-6, wherein the neurotherapeutic is a cognitive enhancer that is selected from a drug-class in the group comprising: cholinesterase inhibitors, glutamatergic molecules, N-methyl d-aspartate (NMDA) receptor antagonists, γ-Aminobutryic acid (GABA) receptor inverse agonists, GABA receptor antagonists, β-secretase inhibitors, α7-nicotinic receptor agonists, serotonin 5-HT₆ receptor antagonists, β1-adrenergic receptor agonists, and monoamine oxidase B (MAO-B) inhibitors.

### Embodiment 8

The pharmaceutical composition of embodiment 7, wherein said cholinesterase inhibitor is selected from the following: donepezil, rivastigmine, galantamine, or huperzine A.

### Embodiment 9

The pharmaceutical composition of embodiment 7, wherein said glutamatergic molecule is an ampakine.

### Embodiment 10

The pharmaceutical composition of embodiment 7, wherein said NMDA receptor antagonist is memantine.

### Embodiment 11

The pharmaceutical composition of embodiment 7, wherein said GABA receptor inverse agonist is a benzodiazepine.

### Embodiment 12

The pharmaceutical composition of embodiment 7, wherein said GABA antagonist is metrazol or bicucilline.

### Embodiment 13

The pharmaceutical composition of embodiment 7, wherein said β-secretase inhibitor is KMI-684.

### Embodiment 14

The pharmaceutical composition of embodiment 7, wherein said α7-nicotinic receptor agonist is EVP-6124 or R3487.

### Embodiment 15

The pharmaceutical composition of embodiment 7, wherein said serotonin 5-HT₆ receptor antagonist is Lu AE58054 or SYN120.

### Embodiment 16

The pharmaceutical composition of embodiment 7, wherein said monoamine oxidase B (MAO-B) inhibitor is RO4602522.

### Embodiment 17

The pharmaceutical composition of any one of embodiments 1-6, wherein said neurotherapeutic is an agent used to treat multiple sclerosis selected from the following: interferon beta-1 a, interferon beta-lb, glatiramer, fingolimod, or natalizumab.

### Embodiment 18

The pharmaceutical composition of any one of embodiments 1-6, wherein said neurotherapeutic is an agent used to treat Parkinson's disease, selected from the following: levodopa, carbidopa, pramipexole, ropinirole, apomorphine, selegiline, rasagiline, benztropine, or amantadine.

### Embodiment 19

The pharmaceutical composition of any one of embodiments 1-6, wherein said neuropathic is an agent used to treat epilepsy selected from the following: felbamate, gabapentin, lamotrigine, topiramate, tiagabine, diazepam, or pregabalin.

### Embodiment 20

The pharmaceutical composition of any one of embodiments 1-6, wherein said neurotherapeutic is an agent used to treat neuropathic pain, selected from the following: naproxen, ibuprofen, or a selective serotonin uptake release inhibitor (SSRI).

### Embodiment 21

A method of treating or reducing the effects of a neurological disorder, the method comprising: administering a therapeutically effective amount of a vitamin D analogue, or a pharmaceutically-acceptable salt thereof, to a subject having symptoms of or diagnosed with a neurological disorder, wherein said effective amount of vitamin D analogue is one that does not exceed a threshold over which hypercalcemia is induced.

### Embodiment 22

The method of embodiment 21, wherein said vitamin D analogue is a vitamin D analogue of the formula:

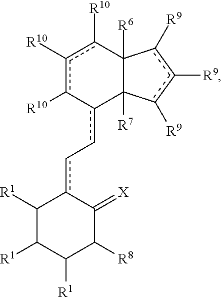

wherein:
each R¹, R⁶, R⁷, R⁸, R⁹ and R¹⁰ is independently H, halo, alkyl, alkenyl, alkynyl, aryl, aralkyl, hydroxyl, sulfhydryl, amino, nitro, cyano, alkoxy, an ester group, an amide group, a carbonate group, a carbamate group, or is a group of the formula:

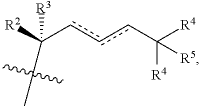

wherein:
each R², R³, R⁴, and R⁵ is independently H, halo, alkyl, aryl, or hydroxyl,
X is C(R¹)₂, O, or NR¹; and
each alkyl, alkenyl, alkynyl, aryl, aralkyl, amino, and alkoxy group is optionally substituted.

### Embodiment 23

The method of embodiment 21, wherein said vitamin D analogue is a vitamin D analogue of the formula:

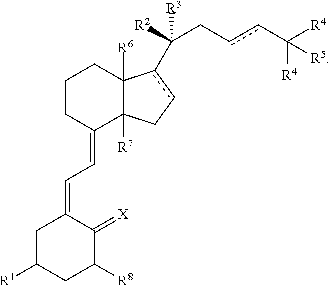

### Embodiment 24

The method of embodiment 23, wherein:
R¹ is H, halo, alkyl, or hydroxyl;
R² is H or alkyl;
R³ is H or alkyl;
each R⁴ is independently H or alkyl;
R⁵ is H, alkyl, or hydroxyl;
R⁶ is H or alkyl;
R⁷ is H or alkyl;
R⁸ is H, halo, alkyl, or hydroxyl; and
X is CH₂ or O.

### Embodiment 25

The method of embodiment 21, wherein said vitamin D analogue is any one of compounds (I-01)-(I-56).

### Embodiment 26

The method of embodiment 21, wherein said vitamin D analogue is elocalcitol

### Embodiment 27

The method of embodiment 26, wherein said threshold is less than or equal to 150 μg per day.

### Embodiment 28

The method of any one of embodiments 21-27, wherein said vitamin D analogue is administered topically, transdermally, intradermally, parenterally, intravenously, intraarterially, subcutaneously, intramuscularly, intracranially, intracolonicly, intraorbitally, ophthalmicly, intraventricularly, intracapsulary, intraspinally, intracisternally, intraperitoneally, intranasally, sublingually, buccally, mucosally, by aerosol, orally, or by suppository.

### Embodiment 29

The method of any one of embodiments 21-27, wherein said vitamin D analogue is administered orally.

### Embodiment 30

The method of any one of embodiments 21-29, wherein said neurological disorder is at least one of the following^{.} Alzheimer's disease, multiple sclerosis, Parkinson's disease, epilepsy, or neuropathic pain.

### Embodiment 31

A method of treating or reducing the effects a neurological disorder, the method comprising: a) administering a therapeutically effective amount of a vitamin D analogue, or a pharmaceutically-acceptable salt thereof, to a subject having symptoms of or diagnosed with a neurological disorder; and b) administering a therapeutically effective amount of at least one neurotherapeutic, or a pharmaceutically-acceptable salt thereof to said subject.

### Embodiment 32

The method of embodiment 31, wherein a dose of said vitamin D analogue is one that does not exceed a threshold over which hypercalcemia is induced.

### Embodiment 33

The method of embodiment 31, wherein said vitamin D analogue is a vitamin D analogue of the formula:

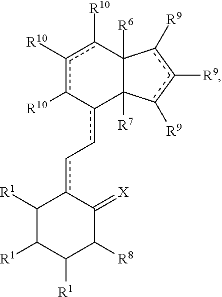

wherein:
each R¹, R⁶, R⁷, R⁸, R⁹ and R¹⁰ is independently H, halo, alkyl, alkenyl, alkynyl, aryl, aralkyl, hydroxyl, sulfhydryl, amino, nitro, cyano, alkoxy, an ester group, an amide group, a carbonate group, a carbamate group, or is a group of the formula:

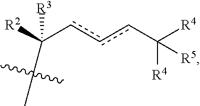

wherein:
each R², R³, R⁴, and R⁵ is independently H, halo, alkyl, aryl, or hydroxyl,
X is C(R¹)₂, O, or NR¹; and
each alkyl, alkenyl, alkynyl, aryl, aralkyl, amino, and alkoxy group is optionally substituted.

### Embodiment 34

The method of embodiment 31, wherein said vitamin D analogue is a vitamin D analogue of the formula:

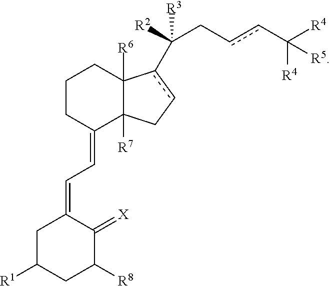

### Embodiment 35

The method of embodiment 34, wherein:
R¹ is H, halo, alkyl, or hydroxyl;
R² is H or alkyl;
R³ is H or alkyl;
each R⁴ is independently H or alkyl;
R⁵ is H, alkyl, or hydroxyl;
R⁶ is H or alkyl;
R⁷ is H or alkyl;
R⁸ is H, halo, alkyl, or hydroxyl; and
X is CH₂ or O.

### Embodiment 36

The method of embodiment 31, wherein said vitamin D analogue is any one of compounds (I-01)-(I-56).

### Embodiment 37

The method of embodiment 31, wherein said vitamin D analogue is elocalcitol.

### Embodiment 38

The method of embodiment 37, wherein said threshold is less than or equal to 150 μg per day of said elocalcitol.

### Embodiment 39

The method of embodiment 31, wherein said neurotherapeutic is a cognitive enhancer that is selected from a drug-class in the group comprising: cholinesterase inhibitors, glutamatergic molecules, N-methyl d-aspartate (NMDA) receptor antagonists, γ-Aminobutryic acid (GABA) receptor inverse agonists, GABA receptor antagonists, β-secretase inhibitors, α7-nicotinic receptor agonists, serotonin 5-HT₆ receptor antagonists, β1-adrenergic receptor agonists, and monoamine oxidase B (MAO-B) inhibitors.

### Embodiment 40

The method of embodiment 39, wherein said cholinesterase inhibitor is selected from the following: donepezil, rivastigmine, galantamine, or huperzine A.

### Embodiment 41

The method of embodiment 39, wherein said glutamatergic molecule is an ampakine.

### Embodiment 42

The method of embodiment 39, wherein said NMDA receptor antagonist is memantine.

### Embodiment 43

The method of embodiment 39, wherein said GABA receptor inverse agonist is a benzodiazepine.

### Embodiment 44

The method of embodiment 39, wherein said GABA antagonist is metrazol or bicucilline.

### Embodiment 45

The method of embodiment 39, wherein said β-secretase inhibitor is KMI-684.

### Embodiment 46

The pharmaceutical composition of embodiment 39, wherein said α7-nicotinic receptor agonist is EVP-6124 or R3487.

### Embodiment 47

The pharmaceutical composition of embodiment 39, wherein said serotonin 5-HT₆ receptor antagonist is Lu AE58054 or SYN120.

### Embodiment 48

The pharmaceutical composition of embodiment 39, wherein said monoamine oxidase B (MAO-B) inhibitor is RO4602522.

### Embodiment 49

The method of embodiment 31, wherein said neurotherapeutic is an agent used to treat multiple sclerosis selected from the following: interferon beta-1a, interferon beta-1b, glatiramer, fingolimod, or natalizumab.

### Embodiment 50

The method of embodiment 31, wherein said neurotherapeutic is an agent used to treat Parkinson's disease, selected from the following: levodopa, carbidopa, pramipexole, ropinirole, apomorphine, selegiline, rasagiline, benztropine, or amantadine.

### Embodiment 51

The method of embodiment 31, wherein said neuropathic is an anti-convulsant agent selected from the following: felbamate, gabapentin, lamotrigine, topiramate, tiagabine, diazepam, or pregabalin.

### Embodiment 52

The method of embodiment 31, wherein said neurotherapeutic is an agent used to treat neuropathic pain, selected from the following: naproxen, ibuprofen, or a selective serotonin uptake release inhibitor (SSRI).

### Embodiment 53

The method of embodiment 31, wherein said vitamin D analogue is administered topically, transdermally, intradermally, parenterally, intravenously, intraarterially, subcutaneously, intramuscularly, intracranially, intracolonicly, intraorbitally, ophthalmicly, intraventricularly, intracapsulary, intraspinally, intracisternally, intraperitoneally, intranasally, sublingually, buccally, mucosally, by aerosol, orally, or by suppository.

### Embodiment 54

The method of embodiment 31, wherein said vitamin D analogue is administered orally.

### Embodiment 55

The method of embodiment 31, wherein said vitamin D analogue and said neurotherapeutic are administered in a single formulation.

### Embodiment 56

The method of embodiment 31, wherein said vitamin D analogue and said neurotherapeutic are administered simultaneously.

### Embodiment 57

The method of embodiment 31, wherein said vitamin D analogue and said neurotherapeutic are administered separately.

### Embodiment 58

The method of embodiment 31, wherein said vitamin D analogue and said neurotherapeutic are synergistic agents and either or both of said vitamin D analogue and at least one said neurotherapeutic have lower therapeutically effective doses when administered in combination than when each agent is administered in the absence of the other.

### Embodiment 59

The method of embodiment 31, wherein the dose of said neurotherapeutic is less than or equal to about 1000 mg.

### Embodiment 60

The method of embodiment 31, wherein the dose of said neurotherapeutic is less than or equal to about 100 mg.

### Embodiment 61

The method of embodiment 31, wherein the dose of said neurotherapeutic is less than or equal to about 10 mg.

### Embodiment 62

The method of embodiment 31, wherein the dose of said neurotherapeutic is less than or equal to about 10 mg.

### Embodiment 63

The method of embodiment 31, wherein the dose of said neurotherapeutic is less than or equal to about 0.1 mg.

### Embodiment 64

The method of embodiment 31, wherein the dose of said neurotherapeutic is less than or equal to about 0.01 mg.

### Embodiment 65

The method of embodiment 31, wherein the dose of said vitamin D analogue is less than or equal to about 1000 mg.

### Embodiment 66

The method of embodiment 31, wherein the dose of said vitamin D analogue is less than or equal to about 100 mg.

### Embodiment 67

The method of embodiment 31, wherein the dose of said vitamin D analogue is less than or equal to about 10 mg.

### Embodiment 68

The method of embodiment 31, wherein the dose of said vitamin D analogue is less than or equal to about 1 mg.

### Embodiment 69

The method of embodiment 31, wherein the dose of said vitamin D analogue is less than or equal to about 0.1 mg.

### Embodiment 70

The method of embodiment 31, wherein the dose of said vitamin D analogue is less than or equal to about 0.01 mg.

### Embodiment 71

The method of any one of embodiments 21 or 31, wherein said neurological disorder is at least one of the following: Alzheimer's disease, multiple sclerosis, Parkinson's disease, epilepsy, or neuropathic pain.

### Embodiment 72

A method for reducing incidence of a neurological disorder, the method comprising: administering a therapeutically effective amount of a vitamin D analogue, or a pharmaceutically-acceptable salt thereof, to a subject at-risk for a neurological disorder, wherein said effective amount of vitamin D analogue is one that does not exceed a threshold over which hypercalcemia is induced.

### Embodiment 73

The method of embodiment 72, wherein the neurological disorder is Parkinson's disease.

### Embodiment 74

The method of embodiment 73, wherein the subject at-risk possesses at least one mutation in at least one gene selected from the group consisting of SNCA, PARK2, PARK8, PINK1, PARK7 ATP13A2 and GBA.

### Embodiment 75

The method of embodiment 72, wherein the neurological disorder is Alzheimer's disease.

### Embodiment 76

The method of embodiment 75, wherein the subject at-risk possesses at least one APOEε4 allele.

### Embodiment 77

The method of any one of embodiments 72-76, wherein the vitamin D analogue is elocalcitol.

### Embodiment 78

The method of any one of embodiments 72-76, wherein the dose of the vitamin D analogue is less than or equal to about 1000 mg.

### Embodiment 79

The method of any one of embodiments 72-76, wherein the dose of said vitamin D analogue is less than or equal to about 100 mg.

### Embodiment 80

The method of any one of embodiments 72-76, wherein the dose of said vitamin D analogue is less than or equal to about 10 mg.

### Embodiment 81

The method of any one of embodiments 72-76, wherein the dose of said vitamin D analogue is less than or equal to about 1 mg.

### Embodiment 82

The method of any one of embodiments 72-76, wherein the dose of said vitamin D analogue is less than or equal to about 0.1 mg.

### Embodiment 83

The method of any one of embodiments 72-76, wherein the dose of said vitamin D analogue is less than or equal to about 0.01 mg.

### Embodiment 84

The method of any one of embodiments 72-76, wherein the method further comprises administering a therapeutically effective amount of a neurotherapeutic, or a pharmaceutically-acceptable salt thereof.

### Embodiment 85

The method of embodiment 84, wherein the dose of said neurotherapeutic is less than or equal to about 1000 mg.

### Embodiment 86

The method of embodiment 84, wherein the dose of said neurotherapeutic is less than or equal to about 100 mg.

### Embodiment 87

The method of embodiment 84, wherein the dose of said neurotherapeutic is less than or equal to about 10 mg.

### Embodiment 88

The method of embodiment 84, wherein the dose of said neurotherapeutic is less than or equal to about 1 mg.

### Embodiment 89

The method of embodiment 84, wherein the dose of said neurotherapeutic is less than or equal to about 0.1 mg.

### Embodiment 90

The method of embodiment 84, wherein the dose of said neurotherapeutic is less than or equal to about 0.01 mg.

### Embodiment 91

The pharmaceutical composition of embodiment 7, wherein the β1-adrenergic receptor agonist is selected from the group consisting of epinephrine, isoproterenol, dobutamine, and xamoterol.

### Embodiment 92

The method of embodiment 39, wherein the β1-adrenergic receptor agonist is selected from the group consisting of epinephrine, isoproterenol, dobutamine, and xamoterol.

What is claimed is:

1. A method of treating or reducing effects of a neurological disorder, the method comprising: administering a therapeutically effective amount of a vitamin D analogue, or a pharmaceutically-acceptable salt thereof, to a subject having symptoms of or diagnosed with a neurological disorder, wherein said effective amount of vitamin D analogue is one that does not exceed a threshold over which hypercalcemia is induced.

2. The method of claim 1, wherein said vitamin D analogue is a vitamin D analogue of the formula: 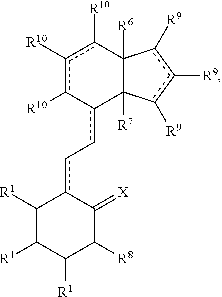
wherein:
each R¹, R⁶, R⁷, R⁸, R⁹ and R¹⁰ is independently H, halo, alkyl, alkenyl, alkynyl, aryl, aralkyl, hydroxyl, sulfhydryl, amino, nitro, cyano, alkoxy, an ester group, an amide group, a carbonate group, a carbamate group, or is a group of the formula: 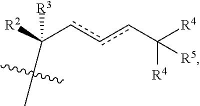
wherein:
each R², R³, R⁴, and R⁵ is independently H, halo, alkyl, aryl, or hydroxyl,
X is C(R¹)₂, O, or NR¹; and
each alkyl, alkenyl, alkynyl, aryl, aralkyl, amino, and alkoxy group is optionally substituted.

3. The method of claim 1, wherein said vitamin D analogue is a vitamin D analogue of the formula:

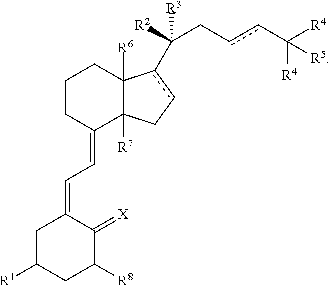

4. The method of claim 3, wherein:
R¹ is H, halo, alkyl, or hydroxyl;
R² is H or alkyl;
R³ is H or alkyl;
each R⁴ is independently H or alkyl;
R⁵ is H, alkyl, or hydroxyl;
R⁶ is H or alkyl;
R⁷ is H or alkyl;
R⁸ is H, halo, alkyl, or hydroxyl; and
X is CH₂ or O.

5. The method of claim 1, wherein said vitamin D analogue is any one of compounds (I-01)-(I-56).

6. The method of claim 1, wherein said vitamin D analogue is elocalcitol

7. The method of claim 6, wherein said threshold is less than or equal to 150 μg per day.

8. The method of claim 1, wherein said vitamin D analogue is administered topically, transdermally, intradermally, parenterally, intravenously, intraarterially, subcutaneously, intramuscularly, intracranially, intracolonicly, intraorbitally, ophthalmicly, intraventricularly, intracapsulary, intraspinally, intracisternally, intraperitoneally, intranasally, sublingually, buccally, mucosally, by aerosol, orally, or by suppository.

9. The method of claim 1, wherein said neurological disorder is at least one of the following: Alzheimer's disease, multiple sclerosis, Parkinson's disease, epilepsy, or neuropathic pain.

10. A pharmaceutical composition comprising:
a) a vitamin D analogue, or a pharmaceutically-acceptable salt thereof;
b) at least one neurotherapeutic, or a pharmaceutically-acceptable salt thereof; and
c) optionally, one or more pharmaceutically-acceptable excipients.

11. The pharmaceutical composition of claim 10, wherein the vitamin D analogue is a vitamin D analogue of the formula: 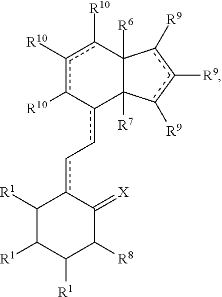
wherein:
each R¹, R⁶, R⁷, R⁸, R⁹ and R¹⁰ is independently H, halo, alkyl, alkenyl, alkynyl, aryl, aralkyl, hydroxyl, sulfhydryl, amino, nitro, cyano, alkoxy, an ester group, an amide group, a carbonate group, a carbamate group, or is a group of the formula: 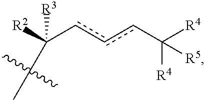
wherein:
each R², R³, R⁴, and R⁵ is independently H, halo, alkyl, aryl, or hydroxyl,
X is C(R¹)₂, O, or NR¹; and
each alkyl, alkenyl, alkynyl, aryl, aralkyl, amino, and alkoxy group is optionally substituted.

12. The pharmaceutical composition of claim 10, wherein the vitamin D analogue is a vitamin D analogue of the formula:

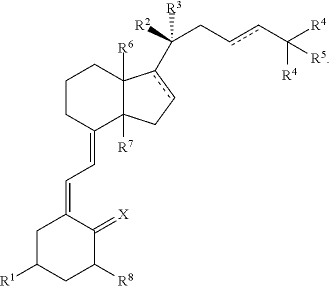

13. The pharmaceutical composition of claim 12, wherein:
R¹ is H, halo, alkyl, or hydroxyl;
R² is H or alkyl;
R³ is H or alkyl;
each R⁴ is independently H or alkyl;
R⁵ is H, alkyl, or hydroxyl;
R⁶ is H or alkyl;
R⁷ is H or alkyl;
R⁸ is H, halo, alkyl, or hydroxyl; and
X is CH₂ or O.

14. The pharmaceutical composition of claim 10, wherein the vitamin D analogue is any one of compounds (I-01)-(I-56).

15. The pharmaceutical composition of claim 10, wherein the vitamin D analogue is elocalcitol.

16. The pharmaceutical composition of claim 10, wherein said neurotherapeutic is a cognitive enhancer that is selected from a drug-class in the group comprising: cholinesterase inhibitors, glutamatergic molecules, N-methyl d-aspartate (NMDA) receptor antagonists, γ-Aminobutryic acid (GABA) receptor inverse agonists, GABA receptor antagonists, β1-secretase inhibitors, α7-nicotinic receptor agonists, serotonin 5-HT₆ receptor antagonists, β1-adrenergic receptor agonists, and monoamine oxidase B (MAO-B) inhibitors.

17. The pharmaceutical composition of claim 16, wherein said cholinesterase inhibitor is selected from the following: donepezil, rivastigmine, galantamine, or huperzine A.

18. The pharmaceutical composition of claim 16, wherein said β1-adrenergic receptor agonist is selected from the group consisting of epinephrine, isoproterenol, dobutamine, and xamoterol.

19. The pharmaceutical composition of claim 16, wherein said NMDA receptor antagonist is memantine.

20. The pharmaceutical composition of claim 16, wherein said α7-nicotinic receptor agonist is EVP-6124 or R3487.

21. The pharmaceutical composition of claim 16, wherein said serotonin 5-HT₆ receptor antagonist is Lu AE58054 or SYN120.

22. The pharmaceutical composition of claim 16, wherein said monoamine oxidase B (MAO-B) inhibitor is RO4602522.

23. The pharmaceutical composition of claim 10, wherein said neurotherapeutic is an agent used to treat multiple sclerosis selected from the following: interferon beta-1a, interferon beta-1b, glatiramer, fingolimod, or natalizumab.

24. The pharmaceutical composition of claim 10, wherein said neurotherapeutic is an agent used to treat Parkinson's disease, selected from the following: levodopa, carbidopa, pramipexole, ropinirole, apomorphine, selegiline, rasagiline, benztropine, or amantadine.

25. A method of treating or reducing effects of a neurological disorder, the method comprising:
a) administering a therapeutically effective amount of a vitamin D analogue, or a pharmaceutically-acceptable salt thereof, to a subject having symptoms of or diagnosed with a neurological disorder; and
b) administering a therapeutically effective amount of at least one neurotherapeutic, or a pharmaceutically-acceptable salt thereof, to said subject.

26. The method of claim 25, wherein a dose of said effective amount of vitamin D analogue is one that does not exceed a threshold over which hypercalcemia is induced.

27. The method of claim 25, wherein said vitamin D analogue is a vitamin D analogue of the formula: 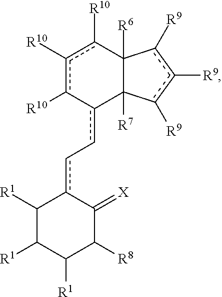
wherein:
each R¹, R⁶, R⁷, R⁸, R⁹ and R¹⁰ is independently H, halo, alkyl, alkenyl, alkynyl, aryl, aralkyl, hydroxyl, sulfhydryl, amino, nitro, cyano, alkoxy, an ester group, an amide group, a carbonate group, a carbamate group, or is a group of the formula: 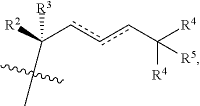
wherein:
each R², R³, R⁴, and R⁵ is independently H, halo, alkyl, aryl, or hydroxyl,
X is C(R¹)₂, O, or NR¹; and
each alkyl, alkenyl, alkynyl, aryl, aralkyl, amino, and alkoxy group is optionally substituted.

28. The method of claim 25, wherein said vitamin D analogue is a vitamin D analogue of the formula:

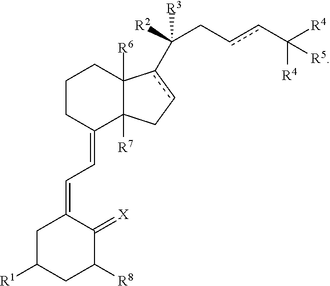

29. The method of claim 28, wherein:
R¹ is H, halo, alkyl, or hydroxyl;
R² is H or alkyl;
R³ is H or alkyl;
each R⁴ is independently H or alkyl;
R⁵ is H, alkyl, or hydroxyl;
R⁶ is H or alkyl;
R⁷ is H or alkyl;
R⁸ is H, halo, alkyl, or hydroxyl; and
X is CH₂ or O.

30. The method of claim 25, wherein said vitamin D analogue is any one of compounds (I-01)-(1-56).

31. The method of claim 25, wherein said vitamin D analogue is elocalcitol.

32. The method of claim 31, wherein said threshold is less than or equal to 150 μg per day of said elocalcitol.

33. The method of claim 25, wherein said neurotherapeutic is a cognitive enhancer that is selected from a drug-class in the group comprising: cholinesterase inhibitors, glutamatergic molecules, N-methyl d-aspartate (NMDA) receptor antagonists, γ-Aminobutryic acid (GABA) receptor inverse agonists, GABA receptor antagonists, β-secretase inhibitors, α7-nicotinic receptor agonists, serotonin 5-HT₆ receptor antagonists, β1-adrenergic receptor agonists, and monoamine oxidase B (MAO-B) inhibitors.

34. The method of claim 33, wherein said cholinesterase inhibitor is selected from the following: donepezil, rivastigmine, galantamine, or huperzine A.

35. The method of claim 33, wherein said β1-adrenergic receptor agonist is selected from the group consisting of epinephrine, isoproterenol, dobutamine, and xamoterol.

36. The method of claim 33, wherein said NMDA receptor antagonist is memantine.

37. The method of claim 33, wherein said a7-nicotinic receptor agonist is EVP-6124 or R3487.

38. The method of claim 33, wherein said serotonin 5-HT₆ receptor antagonist is Lu AE58054 or SYN120.

39. The method of claim 33, wherein said monoamine oxidase B (MAO-B) inhibitor is RO4602522.

40. The method of claim 25, wherein said neurotherapeutic is an agent used to treat multiple sclerosis selected from the following: interferon beta-1a, interferon beta-1b, glatiramer, fingolimod, or natalizumab.

41. The method of claim 25, wherein said neurotherapeutic is an agent used to treat Parkinson's Disease, selected from the following: levodopa, carbidopa, pramipexole, ropinirole, apomorphine, selegiline, rasagiline, benztropine, or amantadine.

42. The method of claim 25, wherein said vitamin D analogue is administered topically, transdermally, intradermally, parenterally, intravenously, intraarterially, subcutaneously, intramuscularly, intracranially, intracolonicly, intraorbitally, ophthalmicly, intraventricularly, intracapsulary, intraspinally, intracisternally, intraperitoneally, intranasally, sublingually, buccally, mucosally, by aerosol, orally, or by suppository.

43. The method of claim 25, wherein said vitamin D analogue and said neurotherapeutic are administered in a single formulation or simultaneously.

44. The method of claim 25, wherein said vitamin D analogue and said neurotherapeutic are administered separately.

45. The method of claim 25, wherein either or both of said vitamin D analogue and at least one said neurotherapeutic have lower therapeutically effective doses when administered in combination than when each agent is administered in the absence of the other.

46. The method of claim 25, wherein the dose of said neurotherapeutic is less than or equal to about 1000, 100, 10, 1, 0.1, or 0.01 mg.

47. The method of any one of claims 1 or 25, wherein the dose of said vitamin D analogue is less than or equal to about 1000, 100, 10, 1, 0.1, or 0.01 mg.

48. The method of any one of claims 1 or 25, wherein said neurological disorder is at least one of the following: Alzheimer's disease, multiple sclerosis, Parkinson's disease, epilepsy, or neuropathic pain.

49. A method for reducing incidence of a neurological disorder, the method comprising: administering a therapeutically effective amount of a vitamin D analogue, or a pharmaceutically-acceptable salt thereof, to a subject at-risk for a neurological disorder, wherein said effective amount of vitamin D analogue is one that does not exceed a threshold over which hypercalcemia is induced.

50. The method of claim 49, wherein the neurological disorder is Parkinson's disease and the subject at-risk possesses at least one mutation in at least one gene selected from the group consisting of SNCA, PARK2, PARK8, PINK1, PARK7 ATP13A2 and GBA.

51. The method of claim 49, wherein the neurological disorder is Alzheimer's disease and the subject at-risk possesses at least one APOEε4 allele.

52. The method of any one of claims 49-51, wherein the vitamin D analogue is elocalcitol.

53. The method of any one of claims 49-51, wherein the method further comprises administering a therapeutically effective amount of a neurotherapeutic, or a pharmaceutically-acceptable salt thereof.
